(19) United States
(12) Patent Application Publication
(10) Pub. No.: US 2026/0308607 A1
(43) Pub. Date: Oct. 8, 2026

(54) ENGINEERING GT2 TYPE OF MEMBRANE PROTEINS FOR TARGETING TO THE TONOPLAST, AND ENGINEERING SORGHUM TO SYNTHESIZE AND DEPOSIT MIXED-LINKAGE GLUCAN (MLG) IN THE VACUOLE

(71) Applicant: Board of Trustees of Michigan State University, East Lansing, MI (US)

(72) Inventors: Federica Brandizzi, Okemos, MI (US); Sang-Jin Kim, Okemos, MI (US)

(21) Appl. No.: 19/481,436

(22) PCT Filed: May 4, 2024
(86) PCT No.: PCT/US2024/027897

§ 371 Date: Nov. 4, 2025

Related U.S. Application Data
(60) Provisional application No. 63/500,086, filed on May 4, 2023.

(51) Int. Cl. C12N 15/82; C12N 9/10

(52) U.S. Cl. CPC C12N 15/8245; C12N 9/1059; C07K 2319/08; C12Y 204/01029

(57) ABSTRACT

Described herein are expression cassettes, plant cells, plant seeds, plants, and methods useful for improving the glucan content and growth of plants.

### PRIORITY

This application claims the benefit of priority of U.S. provisional patent application No. 63/500,086, filed on May 4, 2023, the benefit of priority of which is claimed hereby, and which is incorporated by reference herein in its entirety.

### GOVERNMENT SUPPORT

This invention was made with government support under grant numbers DE-FC02-07ER64494 and DE-SC0018409 awarded by the Department of Energy Great Lakes Bioenergy Research Center. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on May 4, 2024, is name 3000217WO1.xml and is 47,285 bytes in size.

### BACKGROUND

Mixed linkage (1,3;1,4)-β-D-glucan (MLG) are abundant matrix polysaccharides that can occupy up to approximately 40% of the total cell wall in grasses. For example, Brachypodium endosperm can have up to 40% mixed-linkage glucans (Guillon et al. J Exp Bot 62(3):1001-15 (2011)). MLG are pmixedolymers containing β-glucosyl residues with both (1,3)- and (1,4)-linkages. Diverse roles have been suggested for mixed-linkage glucans including regulation of cell growth, cell wall structure and energy storage. The MLG content of grains varies amongst the cereals, with barley, oats and rye having the highest amounts and wheat, maize and rice having relatively low levels.

### SUMMARY

Described herein are plants, plant cells, and plant seeds that provide improved glucan content, as well as methods for making and using such plants, plant cells, and plant seeds. The nucleic acids, expression cassettes, plants, seeds and methods described herein can be used to improve the quality and quantity of plant materials for biofuel production and other uses. Methods of cultivating such plant seeds and plants are also described herein that include, for example, harvesting the plants, seeds, or the tissues of the plants. Such methods can also include isolating glucans, polysaccharides, starch, and/or sugars from the plants, seeds, or tissues of the plants.

For example, plant cells, plant seeds, and plants are described herein that include an expression system comprising at least one expression vector comprising a first nucleic acid segment encoding a vacuolar targeting polypeptide linked in-frame with at least one second nucleic acid segment encoding a cellulose synthase-like Csl protein to form a fusion protein, wherein the vacuolar targeting polypeptide has an activity of localizing the fusion protein to a tonoplast without affecting the protein topology.

In addition, methods are described herein that include comprising growing a plant seed or plant comprising an expression system comprising a heterologous promoter operably linked to a nucleic acid segment encoding a vacuolar targeting polypeptide linked in-frame with a nucleic acid segment encoding a Csl protein to form a fusion protein, to thereby produce a mature plant.

### BRIEF DESCRIPTION OF THE DRAWINGS

FIGS. 1A-B are graphs depicting the regulation of MLG in elongating leaf in plants. FIG. 1A shows there a higher amount of MLG in leaf base versus tip. FIG. 1B shows the similar diurnal variation of MLG in the elongation zone of wild type and the opposite pattern of expression of lichenase responsible for MLG degradation. The graphs of FIGS. 1A-B illustrate that the development and light cycle regulate MLG abundance.

FIG. 2 is an image showing overexpression of CslF6 in Brachypodium results in plants that set seeds and have a shorter stature than wild-type plants.

FIG. 3 is a graph illustrating the amounts of MLG in leaves of Brachypodium that overexpress CslF6 (CslF6OX) (y-axis is leaf MLG μg/mg of AIR). Completion of MLG degradation in leaf was observed at 8 weeks. FIG. 3 shows that Brachypodium tissues that overexpress CslF6 (CslF6OX) have higher mixed-linkage glucan content than wild plant tissues.

FIG. 4 is an image of a Brachypodium plant that expresses CslF6 (CslF6OX), shown at 5 weeks, 6 weeks, and 8 weeks.

FIGS. 5A-B are graphs illustrating that overexpression of Brachypodium CslF6 (BdCSLF6) increases the level of MLG in Brachypodium. FIG. 5A is a graph showing an increase of leaf MLG in Brachypodium isolates overexpressing CslF6 compared to wild-type Brachypodium. FIG. 5B is a graph showing an increase of stem MLG in Brachypodium isolates overexpressing CslF6 compared to wild-type Brachypodium.

FIG. 6 is a graph illustrating the levels of MLG in the transgenic sorghum overexpressing CslF6 genes from Brachypodium (Bd) or Sorghum bicolor (Sb). MLG levels are increased in some transgenic lines compared to wild type plants, with isolates of the transgenic sorghum line overexpressing sorghum CslF6 showing marked increases in MLG production.

FIG. 7 is a graph showing the levels of biomass in transgenic Sorghum bicolor overexpressing BdCslF6 or SbCslF6. Transgenic Sorghum bicolor overproducing MLG grows normally in greenhouse conditions compared to a vector control.

FIG. 8 is a graph showing the stem height and stem biomass of the transgenic sorghum overexpressing CslF6 genes from Brachypodium (Bd) or Sorghum bicolor (Sb). As shown, transgenic sorghum overproducing MLG grows normally in field conditions.

FIG. 9 is a graph showing the levels of MLG in portions of the transgenic sorghum overexpressing CslF6 genes from Brachypodium (Bd) or Sorghum bicolor (Sb) at a boot stage of growth in field conditions during the daytime at 2-4 μm. An increased level of MLG is shown in transgenic lines at the boot stage in daylight.

FIG. 10 is a graph showing the levels of MLG in portions of the transgenic sorghum overexpressing CslF6 genes from Brachypodium (Bd) or Sorghum bicolor (Sb) at a boot stage of growth in field conditions during the nighttime at 5-6 am. An increased level of MLG is shown in each transgenic line at the boot stage at nighttime.

FIGS. 11A-C show the characterization of three sorghum MLG endoglucanases (also called lichenases). FIG. 11A is an illustration of the lichenase-mediated hydrolysis of MLG in plants, causing MLG degradation. FIG. 11B is a graph showing the enzymatic activity of lichenase enzymes in Sorghum. The two Sorghum lichenases have similar enzymatic activity at their optimal condition (near pH 5 and temperature between 25° C. and 45° C.), but one lichenase (SbLCH2) has a lower activity than the other two lichenases (SbLCH1 and SbLCH3). FIG. 11C is an image showing that the three Sorghum enzymes localize in the apoplast, where lichenase activity is expected to take place.

FIG. 12 shows the lichenases' expression pattern during development and light conditions and the lichenase that is likely responsible for MLG degradation in Sorghum.

FIG. 13 is a graph depicting MLG regulation in Sorghum young leaves. High MLG and low starch synthesis were observed in the actively elongating region of the leaves. Fully elongated cells start starch synthesis during the day. MLG degradation is shown not to substantially occur at night and starch is shown to be degraded at night.

FIG. 14 shows images of fluorescence microscopy of Sorghum protoplasts expressing TonoCSLF6 fusion protein. The images show localization of CSLF6 in the tonoplast.

FIG. 15 is a graph showing an increase in MLG in top and bottom portions of Sorghum leaf tops expressing TonoCSLF6 fusion protein.

### DETAILED DESCRIPTION

Described herein are plants, plant cells, and plant seeds that provide improved glucan content, as well as methods for making and using such plants, plant cells, and plant seeds. The nucleic acids, expression cassettes, plants, seeds and methods described herein can be used to improve the quality and quantity of plant materials for biofuel production and other uses. Methods of cultivating such plant seeds and plants are also described herein that include, for example, harvesting the plants, seeds, or the tissues of the plants. Such methods can also include isolating glucans, polysaccharides, starch, and/or sugars from the plants, seeds, or tissues of the plants.

The diets of humans and livestock rely heavily on cereals storage proteins and carbohydrates, including the simple, yet, important, glucose polymer, mixed-linkage glucan (MLG). Storage proteins and the proteins responsible for the production of MLG are synthesized by the endomembrane system.

### CslF6

MLG is a significant cell wall carbohydrate in grasses and an important dietary fiber source for human consumption and biofuel production. MLG biosynthesis depends on the biochemical activity of members of membrane-spanning glycosyltransferase (GT) 2 family of uridine diphosphate glucose (UDP-Glc) utilizing enzymes. The genes responsible for MLG synthesis in grasses share sequence homology to the cellulose synthases (CesAs) and belong to the cellulose synthase-like (Csl) F, H, and J sub-families. CslF6 is the major isoform contributing to total MLG abundance in both vegetative and floral tissues in barley, rice, wheat, and Brachypodium distachyon.

A variety of CslF6 proteins and CslF6 nucleic acids can be used to increase plant glucan content. For example, one sequence of a CslF6 protein from Brachypodium distachyon (Bradi3g16307.1) is shown below as SEQ ID NO:1.

| | |
|---|---|
| 1 | MAPAVAGGSS RGAGCKCGFQ VCVCSGSAAV ASAGSSLEVE |
| 41 | RAMAVTPVEG QAAPVDGESW VGVELGPDGV ETDESGAGVD |
| 81 | DRPVFKTEKI KGVLLHPYRV LIFVRLIAFT LFVIWRISHK |
| 121 | NPDTMWLWVT SICGEFWFGF SWLLDQLPKL NPINRIPDLA |
| 161 | VLRQRFDRAD GTSTLPGLDI FVTTADPIKE PILSTANSVL |
| 201 | SILAADYPVD RNTCYISDDS GMLMTYEAMA ESAKFATLWV |
| 241 | PFCRKHGIEP RGPESYFELK SHPYMGRAHD EFVNDRRRVR |
| 281 | KEYDDFKAKI NSLETDIQQR NDLHNAAVPQ NGDGIPRPTW |
| 321 | MADGVQWQGT WVEPSANHRK GDHAGIVLVL IDHPSHDRLP |
| 361 | GAPASADNAL DFSGVDTRLP MLVYMSREKR PGHNHQKKAG |
| 401 | AMNALTRASA LLSNAPFILN LDCDHYINNS QALRAGICFM |
| 441 | VGRDSDTVAF VQFPQRFEGV DPTDLYANHN RIFFDGTLRA |
| 481 | LDGMQGPIYV GTGCLFRRIT VYGFDPPRIN VGGPCFPALG |
| 521 | GLFAKTKYEK PSMEMTMARA NQAVVPAMAK GKHGFLPLPK |
| 561 | KTYGKSDKFV DTIPRASHPS PYAAEGIRVV DSGAETLAEA |
| 601 | VKVTGSAFEQ KTGWGSELGW VYDTVTEDVV TGYRMHIKGW |
| 641 | RSRYCSIYPH AFIGTAPINL TERLFQVLRW STGSLEIFFS |
| 681 | KNNPLFGSTY LHPLQRVAYI NITTYPFTAI FLIFYTTVPA |
| 721 | LSFVTGHFIV QRPTTMFYVY LGIVLATLLI IAVLEVKWAG |
| 761 | VTVFEWERNG QFWMTASCSA YLAAVCQVLT KVIFRRDISF |
| 801 | KLTSKLPAGD EKKDPYADLY VVRWTPLMIT PIIIIFVNII |
| 841 | GSAVAFAKVL DGEWTHWLKV AGGVFFNFWV LFHLYPFAKG |
| 881 | LLGKHGKTPV VVLVWWAFTF VITAVLYINI PHIHGGGGKH |
| 921 | SVGHGMHHGK KFDGYYLWP |

A nucleotide sequence that encodes the CslF6 protein from Brachypodium distachyon with SEQ ID NO:1 is shown below as SEQ ID NO:2.

| | |
|---|---|
| 1 | ATGGCGCCAG CGGTGGCCGG CGGGAGCAGC CGGGGTGCAG |
| 41 | GGTGTAAGTG CGGGTTCCAG GTGTGCGTGT GCTCTGGGTC |
| 81 | GGCGGCGGTG GCGTCGGCGG GTTCGTCGCT GGAGGTGGAG |
| 121 | AGAGCCATGG CGGTGACGCC GGTGGAAGGG CAGGCGGCGC |
| 161 | CGGTGGACGG CGAGAGCTGG GTCGGCGTCG AGCTCGGCCC |
| 201 | CGACGGCGTG GAGACGGACG AGAGCGGCGC CGGCGTCGAC |
| 241 | GACCGCCCCG TCTTCAAGAC CGAGAAGATC AAGGGCGTCC |
| 281 | TCCTCCACCC CTACAGGGTG CTGATCTTTG TTCGTCTGAT |
| 321 | AGCGTTCACC CTGTTCGTGA TCTGGCGTAT CTCGCACAAG |
| 361 | AACCCGGACA CGATGTGGCT GTGGGTGACC TCCATCTGCG |
| 401 | GCGAGTTCTG GTTCGGCTTC TCCTGGCTGC TGGACCAGCT |
| 441 | TCCAAAGCTC AACCCGATCA ACCGGATCCC GGACCTCGCC |
| 481 | GTGCTCCGGC AACGCTTCGA CCGCGCCGAC GGGACATCCA |
| 521 | CATTGCCGGG CCTCGACATC TTCGTCACCA CGGCCGACCC |
| 561 | CATCAAGGAA CCCATCCTGT CGACGGCCAA CTCCGTGCTC |
| 601 | TCCATCCTGG CCGCCGACTA CCCGGTGGAC CGCAACACCT |
| 641 | GCTACATCTC CGACGACAGC GGCATGCTCA TGACCTACGA |
| 681 | GGCCATGGCG GAGTCGGCCA AGTTCGCCAC CCTCTGGGTG |
| 721 | CCATTCTGCC GCAAGCACGG CATCGAACCA CGCGGGCCGG |
| 761 | AGAGCTACTT CGAGCTCAAG TCGCACCCGT ACATGGGGAG |
| 801 | AGCGCACGAC GAGTTCGTCA ATGACCGCCG CCGGGTGCGC |
| 841 | AAGGAGTATG ATGACTTCAA GGCCAAGATT AACTCTCTGG |
| 881 | AGACTGATAT CCAGCAGAGG AATGATCTGC ATAACGCTGC |
| 921 | CGTGCCGCAG AATGGGGATG GGATCCCCAG GCCTACCTGG |
| 961 | ATGGCTGATG GAGTCCAGTG GCAGGGGACT TGGGTCGAGC |
| 1001 | CGTCCGCTAA TCACCGCAAG GGAGACCACG CCGGCATCGT |
| 1041 | CCTGGTTCTG ATTGACCACC CGAGCCACGA CCGCCTTCCC |
| 1081 | GGCGCGCCGG CGAGCGCCGA CAACGCGCTG GACTTCAGCG |
| 1121 | GCGTGGACAC CCGCCTCCCG ATGCTCGTCT ACATGTCCCG |
| 1161 | CGAGAAGCGC CCAGGCCACA ACCACCAGAA GAAGGCCGGC |
| 1201 | GCCATGAACG CGCTCACCAG GGCTTCCGCG CTGCTCTCCA |
| 1241 | ACGCGCCCTT CATCCTCAAC CTCGACTGCG ACCACTACAT |
| 1281 | CAACAACTCC CAGGCCCTCC GCGCCGGGAT CTGCTTCATG |
| 1321 | GTCGGCCGGG ACAGCGACAC CGTCGCCTTC GTGCAGTTCC |
| 1361 | CGCAGCGGTT CGAGGGCGTC GACCCCACGG ACCTCTACGC |
| 1401 | CAACCACAAC CGCATCTTCT TCGACGGCAC CCTCAGGGCG |
| 1441 | CTCGACGGAA TGCAAGGCCC GATCTATGTC GGCACGGGAT |
| 1481 | GCCTCTTCCG GCGCATCACC GTCTACGGCT TCGACCCGCC |
| 1521 | CAGGATCAAC GTCGGCGGGC CATGCTTCCC TGCTCTCGGT |
| 1561 | GGCCTGTTCG CCAAGACCAA GTATGAGAAG CCCAGCATGG |
| 1601 | AGATGACCAT GGCGAGAGCC AACCAGGCCG TGGTGCCGGC |
| 1641 | CATGGCCAAG GGGAAGCACG GCTTCCTGCC GCTCCCCAAG |
| 1681 | AAGACGTACG GGAAGTCCGA CAAGTTCGTG GACACCATCC |
| 1721 | CGCGCGCGTC CCACCCGTCG CCGTACGCGG CGGAGGGGAT |
| 1761 | CCGCGTGGTG GACTCCGGCG CGGAGACTCT GGCTGAGGCC |
| 1801 | GTCAAGGTGA CCGGATCGGC ATTCGAGCAG AAGACCGGAT |
| 1841 | GGGGCAGCGA GCTCGGCTGG GTCTACGACA CTGTCACAGA |
| 1881 | GGACGTGGTG ACTGGCTACA GGATGCACAT CAAGGGCTGG |
| 1921 | AGGTCCCGCT ACTGCTCCAT CTACCCGCAC GCCTTCATCG |
| 1961 | GCACCGCCCC GATCAACCTC ACGGAGCGGC TCTTCCAGGT |
| 2001 | GCTCCGCTGG TCCACCGGCT CCCTCGAGAT CTTCTTCTCC |
| 2041 | AAGAACAACC CGCTCTTCGG CAGCACCTAC CTGCACCCGC |
| 2081 | TCCAGCGCGT CGCCTACATC AACATCACCA CATACCCGTT |
| 2121 | CACCGCCATC TTCCTCATCT TCTACACCAC CGTGCCGGCG |
| 2161 | CTCTCCTTCG TCACCGGCCA CTTCATCGTG CAGCGCCCGA |
| 2201 | CGACCATGTT CTACGTCTAC CTGGGGATCG TGCTGGCGAC |
| 2241 | GCTGCTCATC ATCGCTGTTC TTGAGGTCAA GTGGGCTGGA |
| 2281 | GTGACAGTGT TCGAGTGGTT CAGGAACGGG CAGTTCTGGA |
| 2321 | TGACGGCTAG CTGCTCCGCC TACCTTGCTG CTGTGTGCCA |
| 2361 | GGTGCTCACC AAGGTGATCT TCAGGAGGGA CATCTCATTC |
| 2401 | AAGCTCACTT CCAAGCTGCC TGCTGGGGAC GAGAAGAAGG |
| 2441 | ACCCCTATGC CGATCTGTAC GTGGTGCGTT GGACTCCACT |
| 2481 | CATGATCACT CCAATCATCA TCATCTTCGT CAACATCATC |
| 2521 | GGCTCGGCGG TGGCCTTCGC CAAGGTGCTG GACGGCGAGT |
| 2561 | GGACGCACTG GCTCAAGGTG GCGGGAGGAG TCTTCTTCAA |
| 2601 | CTTCTGGGTG CTGTTCCACC TCTACCCGTT CGCCAAGGGT |
| 2641 | CTCCTGGGGA AGCATGGCAA GACCCCCGTC GTCGTGCTCG |
| 2681 | TCTGGTGGGC ATTCACCTTC GTCATCACCG CCGTCCTCTA |
| 2721 | CATCAACATC CCGCACATCC ATGGAGGAGG AGGCAAGCAC |
| 2761 | AGCGTGGGGC ATGGGATGCA CCATGGCAAG AAGTTCGACG |
| 2801 | GCTACTACCT CTGGCCGTGA |

A nucleotide sequence that encodes the CslF6 protein from Brachypodium distachyon with SEQ ID NO:1 and that has been codon-optimized for expression in Brachypodium distachyon is shown below as SEQ ID NO:3.

| | |
|---|---|
| 1 | ATGGCTCCAG CTGTTGCTGG CGGCTCCTCT AGGGGCGCTG |
| 41 | GCTGCAAGTG CGGCTTCCAG GTGTGCGTGT GCTCCGGCTC |
| 81 | TGCCGCCGTG GCCTCCGCCG GCTCATCCCT CGAGGTCGAG |
| 121 | AGGGCCATGG CTGTTACCCC AGTTGAGGGC CAGGCCGCTC |
| 161 | CAGTGGACGG CGAGTCCTGG GTGGGCGTTG AGCTTGGCCC |
| 201 | AGACGGCGTC GAGACCGACG AGTCCGGCGC TGGCGTGGAC |
| 241 | GACAGGCCAG TGTTCAAGAC CGAGAAGATC AAGGGCGTGC |
| 281 | TCCTCCACCC ATACAGGGTG CTCATCTTCG TGAGGCTGAT |
| 321 | CGCCTTCACC CTCTTCGTGA TCTGGCGCAT CTCCCACAAG |
| 361 | AACCCGGACA CCATGTGGCT CTGGGTGACC TCTATTTGCG |
| 401 | GCGAGTTCTG GTTCGGCTTC TCCTGGCTCC TCGACCAGCT |
| 441 | CCCAAAGCTC AACCCGATCA ACCGCATCCC AGATCTCGCC |
| 481 | GTTCTCAGGC AGAGGTTCGA TAGGGCCGAC GGCACCTCCA |
| 521 | CCCTCCCAGG CCTTGATATT TTCGTGACCA CCGCCGACCC |
| 561 | CATCAAGGAG CCAATTCTCT CAACCGCCAA CTCCGTGCTC |
| 601 | TCTATCCTCG CCGCCGATTA CCCGGTGGAT AGGAACACGT |
| 641 | GCTACATCTC CGACGACAGC GGCATGCTCA TGACCTACGA |
| 681 | GGCTATGGCC GAGTCCGCCA AGITCGCTAC CCTCTGGGTG |
| 721 | CCATTCTGCC GCAAGCACGG CATCGAGCCA AGGGGCCCAG |
| 761 | AGTCCTACTT CGAGCTTAAG TCCCACCCGT ACATGGGCAG |
| 801 | GGCCCATGAC GAGTTCGTGA ACGATAGGCG CAGGGTGAGG |
| 841 | AAGGAGTACG ACGACTTCAA GGCCAAGATC AACTCCCTCG |
| 881 | AGACGGACAT CCAGCAGAGG AACGACCTCC ATAACGCCGC |
| 921 | CGTGCCACAG AACGGGGACG GCATCCCAAG GCCAACCTGG |
| 961 | ATGGCCGATG GCGTGCAGTG GCAGGGCACC TGGGTTGAGC |
| 1001 | CATCTGCCAA CCATAGGAAG GGCGATCACG CCGGCATTGT |
| 1041 | GCTCGTGCTC ATCGACCATC CATCCCACGA CAGGCTCCCA |
| 1081 | GGCGCCCCAG CCTCTGCCGA CAACGCCCTC GACTTCTCCG |
| 1121 | GCGTGGACAC CAGGCTTCCA ATGCTCGITT ACATGTCCCG |
| 1161 | CGAGAAGAGG CCAGGCCACA ACCACCAGAA GAAGGCTGGC |
| 1201 | GCTATGAACG CCCTTACCAG GGCTTCTGCT CTCCTCTCCA |
| 1241 | ACGCCCCGTT CATCCTCAAC CTCGACTGCG ACCACTACAT |
| 1281 | CAACAACAGC CAGGCTCTCA GGGCCGGCAT CTGCTTCATG |
| 1321 | GTGGGCAGGG ATTCTGACAC CGTGGCCTTC GTTCAGTTCC |
| 1361 | CGCAGCGCTT CGAGGGGGTT GACCCAACCG ATCTCTACGC |
| 1401 | CAACCACAAC AGGATTTTCT TCGATGGCAC CCTCAGGGCC |
| 1441 | CTCGATGGCA TGCAGGGCCC TATCTACGTG GGCACCGGCT |
| 1481 | GCCTCTTCAG GCGCATCACC GTGTACGGCT TCGACCCGCC |
| 1521 | AAGGATTAAC GTTGGCGGCC CATGCTTCCC AGCTCTCGGC |
| 1561 | GGCCTCTTCG CTAAGACCAA GTACGAGAAG CCCAGCATGG |
| 1601 | AGATGACCAT GGCCAGGGCC AACCAGGCCG TTGTTCCAGC |
| 1641 | TATGGCTAAG GGGAAGCACG GCTTCCTGCC ACTCCCGAAG |
| 1681 | AAGACCTACG GCAAGAGCGA CAAGTTCGTC GACACCATTC |
| 1721 | CAAGGGCCTC CCACCCATCT CCATACGCTG CCGAGGGCAT |
| 1761 | TAGGGTTGTG GACTCTGGCG CCGAGACCCT CGCCGAGGCC |
| 1801 | GTGAAGGTGA CCGGCTCCGC CTTCGAGCAG AAGACCGGCT |
| 1841 | GGGGCTCCGA GCTTGGCTGG GTTTACGACA CCGTGACCGA |
| 1881 | GGATGTGGTC ACCGGCTACA GGATGCACAT TAAGGGCTGG |
| 1921 | CGCAGCAGGT ACTGCTCCAT CTACCCACAT GCCTTCATCG |
| 1961 | GCACCGCCCC CATTAACCTC ACCGAGAGGC TTTTCCAGGT |
| 2001 | GCTCAGGTGG TCTACCGGCA GCCTCGAGAT CTTCTTCAGC |
| 2041 | AAGAACAACC CGCTGTTCGG CTCCACCTAC CTGCATCCAC |
| 2081 | TCCAGAGGGT GGCCTACATT AACATCACCA CCTACCCGTT |
| 2121 | CACCGCCATC TTCCTCATCT TCTACACGAC CGTGCCCGCC |
| 2161 | CTCTCATTCG TGACCGGCCA TTTCATTGTG CAGAGGCCGA |
| 2201 | CCACCATGTT CTACGTGTAC CTCGGGATCG TGCTCGCCAC |
| 2241 | CCTCCTCATT ATTGCCGTGC TCGAGGTTAA GTGGGCTGGC |
| 2281 | GTGACCGTGT TCGAGTGGTT CCGCAACGGC CAGTTCTGGA |
| 2321 | TGACCGCCTC TTGCTCTGCT TACCTCGCCG CTGTTTGCCA |
| 2361 | GGTCCTCACC AAGGTTATCT TCCGCAGGGA CATCTCCTTC |
| 2401 | AAGCTCACCT CCAAGCTCCC AGCCGGCGAC GAGAAGAAGG |
| 2441 | ACCCATACGC CGATCTGTAC GTGGTGAGGT GGACCCCGCT |
| 2481 | CATGATCACC CCGATCATCA TCATTTTCGT CAACATCATC |
| 2521 | GGCTCCGCGG TCGCCTTCGC CAAGGTGCTC GATGGCGAGT |
| 2561 | GGACCCATTG GCTTAAGGTC GCCGGCGGCG TGTTCTTCAA |
| 2601 | CTTCTGGGTT CTCTTCCACC TCTACCCTTT CGCGAAGGGC |
| 2641 | CTTCTTGGCA AGCACGGCAA GACCCCAGTG GTGGTTCTTG |
| 2681 | TCTGGTGGGC CTTCACCTTC GTCATCACCG CCGTGCTGTA |
| 2721 | CATCAACATC CCGCACATCC ATGGCGGCGG CGGCAAGCAC |
| 2761 | TCCGTGGGCC ACGGCATGCA CCATGGCAAG AAGTTCGACG |
| 2801 | GCTACTACCT CTGGCCGTGA |

CslF6 proteins and nucleic acids from a variety of species can be used in the plants, seeds, plant cells and methods described herein. For example, a CslF6 amino acid sequence from wheat (Triticum aestivum) can be used that has about 86% sequence identity with the CslF6 from Brachypodium distachyon that has SEQ ID NO:1. This wheat CslF6 sequence is shown below with SEQ ID NO:5.

| | |
|---|---|
| 1 | MAPAVAGGGR VRSNEPAAAA TAPASGKPCV CGFQVCACTG |
| 41 | SAAVASAASS LDMDIVAMGQ IGAVNDESWV GVELGEDGET |
| 81 | DESGAAVDDR PVFRTEKIKG VLLHPYRVLI FVRLIAFTLF |
| 121 | VIWRISHKNP DAMWLWVTSI CGEFWFGFSW LLDQLPKLNP |
| 161 | INRVPDLAVL RORFDRPDGT STLPGLDIFV TTADPIKEPI |
| 201 | LSTANSVLSI LAADYPVDRN TCYVSDDSGM LLTYEALAES |
| 241 | SKFATLWVPF CRKHGIEPRG PESYFELKSH PYMGRAQDEF |
| 281 | VNDRRRVRKE YDEFKARINS LEHDIKQRND GYNAANAHRE |
| 321 | GEPRPTWMAD GTQWEGTWVD ASENHRRGDH AGIVLVLLNH |
| 361 | PSHRRQTGPP ASADNPLDFS GVDVRLPMLV YMSREKRPGH |
| 401 | DHOKKAGAMN ALTRASALLS NSPFILNLDC NHYINNSQAL |
| 441 | RAGICFMVGR DSDTVAFVQF PORFEGVDPT DLYANHNRIF |
| 481 | FDGTLRALDG MQGPIYVGTG CLFRRITVYG FDPPRINVGG |
| 521 | PCFPRLAGLF AKTKYEKPGL EMTMAKAKAA PVPAKGKHGF |
| 561 | LPLPKKTYGK SDAFVDSIPR ASHPSPYAAA AEGIVADEAT |
| 601 | IVEAVNVTAA AFEKKTGWGK EIGWVYDTVT EDVVTGYRMH |
| 641 | IKGWRSRYCS IYPHAFIGTA PINLTERLFQ VLRWSTGSLE |
| 681 | IFFSKNNPLF GSTYLHPLQR VAYINITTYP FTAIFLIFYT |
| 721 | TVPALSFVTG HFIVQRPTTM FYVYLGIVLS TLLVIAVLEV |
| 761 | KWAGVTVFEW FRNGOFWMTA SCSAYLAAVC QVLTKVIFRR |
| 801 | DISFKLTSKL PSGDEKKDPY ADLYVVRWTP LMITPIIIIF |
| 841 | VNIIGSAVAF AKVLDGEWTH WLKVAGGVFF NFWVLFHLYP |
| 881 | FAKGILGKHG KTPVVVLVWW AFTFVITAVF YINIPHMHSS |
| 921 | GGKHTTVHGH HGKKFVDAGY YNWP |

A nucleotide sequence that encodes the CslF6 protein from Triticum aestivum with SEQ ID NO:5 is shown below as SEQ ID NO:6.

| | |
|---|---|
| 1 | ATGGCGCCAG CGGTGGCCGG AGGAGGCCGC GTGCGGAGCA |
| | ACGAGCCGGC TGCTGCCGCT |
| 61 | ACCGCGCCCG CCAGCGGGAA GCCCTGCGTC TGCGGCTTCC |
| | AGGTGTGCGC CTGCACGGGA |
| 121 | TCGGCCGCGG TGGCGTCTGC CGCCTCCTCG CTCGACATGG |
| | ACATCGTGGC CATGGGGCAG |
| 181 | ATAGGCGCCG TCAACGACGA GAGCTGGGTG GGCGTGGAGC |
| | TCGGCGAGGA CGGCGAGACC |
| 241 | GACGAAAGCG GTGCCGCCGT TGACGACCGC CCCGTCTTCC |
| | GCACCGAGAA AATCAAGGGT |
| 301 | GTCCTCCTCC ACCCCTACCG GGTGCTGATC TTCGTCCGTC |
| | TCATCGCCTT CACCCTGTTC |
| 361 | GTGATCTGGC GTATTTCCCA CAAGAACCCT GACGCCATGT |
| | GGCTGTGGGT GACATCCATC |
| 421 | TGCGGCGAGT TCTGGTTCGG TTTCTCCTGG CTGCTGGATC |
| | AGCTCCCCAA GCTGAACCCC |
| 481 | ATCAACCGCG TGCCGGACCT GGCCGTGCTG CGGCAGCGCT |
| | TCGACCGCCC CGACGGCACC |
| 541 | TCCACCCICC CGGGGCTGGA CATCTTCGTC ACCACGGCCG |
| | ACCCCATCAA GGAGCCCATC |
| 601 | CTCTCCACTG CCAACTCGGT GCTCTCCATC CTCGCCGCCG |
| | ACTACCCCGT CGACCGCAAC |
| 661 | ACATGCTACG TTTCTGACGA CAGTGGCATG CTGCTCACCT |
| | ACGAGGCCCT GGCTGAGTCC |
| 721 | TCCAAGTTCG CCACCCTCTG GGTGCCCTTC TGCCGCAAGC |
| | ACGGGATCGA GCCCAGGGGC |
| 781 | CCGGAGAGCT ACTTTGAGCT CAAGTCCCAC CCCTACATGG |
| | GGAGAGCCCA GGACGAGTTC |
| 841 | GIGAACGACC GTCGCCGCGT CCGCAAGGAG TACGACGAGT |
| | TCAAGGCCAG GATCAACAGC |
| 901 | CTGGAGCATG ACATCAAGCA GCGCAACGAC GGGTACAACG |
| | CCGCCAACGC CCACCGGGAA |
| 961 | GGCGAGCCCC GACCCACCTG GATGGCCGAC GGCACCCAGT |
| | GGGAGGGCAC CTGGGTCGAC |
| 1021 | GCCTCCGAGA ACCACCGCAG GGGCGACCAC GCCGGCATCG |
| | TCCTGGTGCT GCTGAACCAC |
| 1081 | CCGAGCCACC GCCGGCAGAC GGGCCCGCCG GCGAGTGCTG |
| | ACAACCCACT GGACTTCAGC |
| 1141 | GGCGTGGATG TGCGTCTCCC CATGCTGGTG TACGTGTCCC |
| | GTGAGAAGCG CCCCGGACAC |
| 1201 | GACCACCAGA AGAAGGCCGG CGCCATGAAC GCGCTCACCC |
| | GCGCCTCCGC GCTGCTCTCC |
| 1261 | AACTCCCCCT TCATCCTCAA CCTCGACTGC GACCACTACA |
| | TCAACAACTC CCAGGCCCTC |
| 1321 | CGCGCCGGCA TCTGCTTCAT GGTGGGACGC GACAGCGACA |
| | CCGTCGCCTT CGTCCAGTTC |
| 1381 | CCGCAGCGCT TCGAGGGCGT CGACCCCACC GACCTCTACG |
| | CCAACCACAA CCGCATCTTC |
| 1441 | TTCGACGGCA CCCTCCGTGC CCTCGACGGC ATGCAGGGCC |
| | CCATCTACGT CGGCACCGGC |
| 1501 | TGTCTCTTCC GCCGCATCAC CGTCTACGGC TTCGACCCGC |
| | CCAGGATCAA CGTCGGCGGG |
| 1561 | CCCTGCTTCC CCAGGCTCGC CGGGCTCTTC GCCAAGACCA |
| | AGTACGAGAA GCCTGGGCTC |
| 1621 | GAGATGACCA TGGCCAAGGC CAAGGCTGCG CCGGTGCCCG |
| | CCAAGGGCAA GCACGGCTTC |
| 1681 | CTGCCTCTGC CCAAGAAGAC GTACGGCAAG TCGGACGCCT |
| | TCGTGGACAG CATCCCGCGC |
| 1741 | GCGTCGCACC CGTCGCCTTA CGCCGCGGCG GCTGAGGGCA |
| | TCGTGGCCGA CGAGGCGACC |
| 1801 | ATCGTGGAGG CGGTGAACGT GACGGCGGCG GCGTTCGAGA |
| | AGAAGACCGG GTGGGGCAAA |
| 1861 | GAGATCGGCT GGGTGTACGA CACCGTGACG GAGGACGTGG |
| | TGACCGGGTA CCGGATGCAT |
| 1921 | ATCAAGGGGT GGCGGTCACG CTACTGCTCC ATCTACCCAC |
| | ACGCCTTCAT CGGCACCGCA |
| 1981 | CCCATCAACC TCACGGAGAG GCTCTTCCAG GTGCTTCGCT |
| | GGTCCACGGG CTCCCTCGAG |
| 2041 | ATCTTCTTCT CCAAGAACAA CCCGCTCTTC GGCAGCACCT |
| | ACCTCCACCC GCTGCAGCGC |
| 2101 | GTCGCCTACA TCAACATCAC CACCTACCCG TTCACCGCCA |
| | TCTTCCTCAT CTTCTACACC |
| 2161 | ACCGTGCCGG CGCTCTCCTT CGTCACTGGC CACTTCATCG |
| | TGCAGCGCCC CACCACCATG |
| 2221 | TTCTACGTCT ACCTGGGCAT CGTGCTCTCC ACGCTGCTCG |
| | TCATCGCCGT GCTGGAGGTC |
| 2281 | AAGTGGGCCG GGGTCACCGT CTTCGAGTGG TTCAGGAACG |
| | GCCAGTTCTG GATGACGGCA |
| 2341 | AGTTGCTCCG CCTACCTCGC CGCCGTGTGC CAGGTGCTCA |
| | CCAAGGTGAT ATTCCGGCGT |
| 2401 | GACATCTCCT TCAAGCTCAC ATCCAAGCTA CCGTCGGGAG |
| | ACGAGAAGAA GGACCCCTAC |
| 2461 | GCCGACCTCT ACGTGGTGCG CTGGACGCCG CTCATGATCA |
| | CACCCATCAT CATCATTTTC |
| 2521 | GTCAACATCA TCGGGTCGGC GGTGGCCTTC GCCAAGGTGC |
| | TCGACGGCGA GTGGACGCAC |
| 2581 | TGGCTCAAGG TCGCCGGCGG CGTCTTCTTC AACTTCTGGG |
| | TGCTCTTCCA CCTCTACCCG |
| 2641 | TTCGCCAAGG GCATCCTGGG GAAGCACGGC AAGACGCCAG |
| | TCGTGGTGCT CGTCTGGTGG |
| 2701 | GCATTCACCT TCGTCATCAC CGCCGTGCTC TACATCAACA |
| | TCCCCCACAT GCATAGCTCG |
| 2761 | GGAGGCAAGC ACACAACGGT GCATGGTCAC CATGGCAAGA |
| | AGTTCGTCGA CGCAGGGTAC |
| 2821 | TACAACTGGC CGTGAGGTCC CTGTTGACGA CTTTGCCGCC |
| | GCGCATCCGG ACAAGACAAC |
| 2881 | GACGACCTGC GACAAGAAAC AACAAGTCAT CCACTGAACA |
| | GTGCA |

A CslF6 amino acid sequence from barley (Hordeum vulgare) has about 86% sequence identity with the CslF6 from Brachypodium distachyon that has SEQ ID NO:1. This barley CslF6 sequence is shown below with SEQ ID NO:7.

| | |
|---|---|
| 1 | MAPAVAGGGR VRSNEPVAAA AAAPAASGKP CVCGFQVCAC |
| 41 | TGSAAVASAA SSLDMDIVAM GQIGAVNDES WVGVELGEDG |
| 81 | ETDESGAAVD DRPVFRTEKI KGVLLHPYRV LIFVRLIAFT |
| 121 | LFVIWRISHK NPDAMWLWVT SICGEFWFGF SWLLDQLPKL |
| 161 | NPINRVPDLA VLRQRFDRPD GTSTLPGLDI FVTTADPIKE |
| 201 | PILSTANSVL SILAADYPVD RNTCYVSDDS GMLLTYEALA |
| 241 | ESSKFATLWV PFCRKHGIEP RGPESYFELK SHPYMGRAQD |
| 281 | EFVNDRRRVR KEYDEFKARI NSLEHDIKQR NDGYNAAIAH |
| 321 | SQGVPRPTWM ADGTQWEGTW VDASENHRRG DHAGIVLVLL |
| 361 | NHPSHRRQTG PPASADNPLD LSGVDVRLPM LVYVSREKRP |
| 401 | GHDHQKKAGA MNALTRASAL LSNSPFILNL DCDHYINNSQ |
| 441 | ALRAGICFMV GRDSDTVAFV QFPQRFEGVD PTDLYANHNR |
| 481 | IFFDGTLRAL DGMQGPIYVG TGCLFRRITV YGFDPPRINV |
| 521 | GGPCFPRLAG LFAKTKYEKP GLEMTTAKAK AAPVPAKGKH |
| 561 | GFLPLPKKTY GKSDAFVDTI PRASHPSPYA AAAEGIVADE |
| 601 | ATIVEAVNVT AAAFEKKIGW GKEIGWVYDT VTEDVVTGYR |
| 641 | MHIKGWRSRY CSIYPHAFIG TAPINLTERL FQVLRWSTGS |
| 681 | LEIFFSKNNP LFGSTYLHPL QRVAYINITT YPFTAIFLIF |
| 721 | YTTVPALSFV IGHFIVQRPT TMFYVYLGIV LSTLLVIAVL |
| 761 | EVKWAGVTVF EWFRNGQFWM TASCSAYLAA VCQVLTKVIF |
| 801 | RRDISFKLTS KLPSGDEKKD PYADLYVVRW TPLMITPIII |
| 841 | IFVNIIGSAV AFAKVLDGEW THWLKVAGGV FFNFWVLFHL |
| 881 | YPFAKGILGK HGKTPVVVLV WWAFTEVITA VLYINIPHMH |
| 921 | TSGGKHTTVH GHHGKKLVDT GLYGWLH |

A nucleotide sequence that encodes the CslF6 protein from Hordeum vulgare with SEQ ID NO:7 is shown below as SEQ ID NO:8.

| | |
|---|---|
| 1 | cgtaaaggag agtgagtgcg tgcattgagg acgacggcca |
| | tggcgccagc ggtggccgga |
| 61 | gggggccgcg tgcggagcaa tgagccggtt gctgctgctg |
| | ccgccgcgcc ggcggccagc |
| 121 | ggcaagccct gcgtgtgcgg cttccaggtt tgcgcctgca |
| | cggggtcggc cgcggtggcc |
| 181 | tccgccgcct cgtcgctgga catggacatc gtggccatgg |
| | ggcagatcgg cgccgtcaac |
| 241 | gacgagagct gggtgggcgt ggagctcggc gaagatggcg |
| | agaccgacga aagcggtgcc |
| 301 | gccgttgacg accgccccgt attccgcacc gagaagatca |
| | agggtgtcct cctccacccc |
| 361 | taccgggtgc tgattttcgt tcgtctgatc gccttcacgc |
| | tgttcgtgat ctggcgtatc |
| 421 | tcccacaaga acccagacgc gatgtggctg tgggtgacat |
| | ccatctgcgg cgagttctgg |
| 481 | ttcggtttct cgtggctgct agatcagctg cccaagctga |
| | accccatcaa ccgcgtgccg |
| 541 | gacctggcgg tgctgcggca gcgcttcgac cgccccgacg |
| | gcacctccac gctcccgggg |
| 601 | ctggacatct tcgtcaccac ggccgacccc atcaaggagc |
| | ccatcctctc caccgccaac |
| 661 | tcggtgctct ccatcctggc cgccgactac cccgtggacc |
| | gcaacacatg ctacgtctcc |
| 721 | gacgacagtg gcatgctgct cacctacgag gccctggcag |
| | agtcctccaa gttcgccacg |
| 781 | ctctgggtgc ccttctgccg caagcacggg atcgagccca |
| | ggggtccgga gagctacttc |
| 841 | gagctcaagt cacaccctta catggggaga gcccaggacg |
| | agttcgtcaa cgaccgccgc |
| 901 | cgcgttcgca aggagtacga cgagttcaag gccaggatca |
| | acagcctgga gcatgacatc |
| 961 | aagcagcgca acgacgggta caacgccgcc attgcccaca |
| | gccaaggcgt gccccggccc |
| 1021 | acctggatgg cggacggcac ccagtgggag ggcacatggg |
| | tcgacgcctc cgagaaccac |
| 1081 | cgcaggggcg accacgccgg catcgtactg gtgctgctga |
| | accacccgag ccaccgccgg |
| 1141 | cagacgggcc cgccggcgag cgctgacaac ccactggact |
| | tgagcggcgt ggatgtgcgt |
| 1201 | ctccccatgc tggtgtacgt gtcccgtgag aagcgccccg |
| | ggcacgacca ccagaagaag |
| 1261 | gccggtgcca tgaacgcgct tacccgcgcc tcggcgctgc |
| | tctccaactc ccccttcatc |
| 1321 | ctcaacctcg actgcgatca ttacatcaac aactcccagg |
| | cccttcgcgc cggcatctgc |
| 1381 | ttcatggtgg gacgggacag cgacacggtt gccttcgtcc |
| | agttcccgca gcgcttcgag |
| 1441 | ggcgtcgacc ccaccgacct ctacgccaac cacaaccgca |
| | tcttcttcga cggcaccctc |
| 1501 | cgtgccctgg acggcatgca gggccccatc tacgtcggca |
| | ctgggtgtct cttccgccgc |
| 1561 | atcaccgtct acggcttcga cccgccgagg atcaacgtcg |
| | gcggtccctg cttccccagg |
| 1621 | ctcgccgggc tcttcgccaa gaccaagtac gagaagcccg |
| | ggctcgagat gaccacggcc |
| 1681 | aaggccaagg ccgcgcccgt gcccgccaag ggtaagcacg |
| | gcttcttgcc actgcccaag |
| 1741 | aagacgtacg gcaagtcgga cgccttcgtg gacaccatcc |
| | cgcgcgcgtc gcacccgtcg |
| 1801 | ccctacgccg cggcggctga ggggatcgtg gccgacgagg |
| | cgaccatcgt cgaggcggtg |
| 1861 | aacgtgacgg ccgccgcgtt cgagaagaag accggctggg |
| | gcaaagagat cggctgggtg |
| 1921 | tacgacaccg tcacggagga cgtggtcacc ggctaccgga |
| | tgcatatcaa ggggtggcgg |
| 1981 | tcacgctact gctccatcta cccacacgcc ttcatcggca |
| | ccgcccccat caacctcacg |
| 2041 | gagaggctct tccaggtgct ccgctggtcc acgggatccc |
| | tcgagatctt cttctccaag |
| 2101 | aacaacccgc tcttcggcag cacatacctc cacccgctgc |
| | agcgcgtcgc ctacatcaac |
| 2161 | atcaccactt accccttcac cgccatcttc ctcatcttct |
| | acaccaccgt gccggcgcta |
| 2221 | tccttcgtca ccggccactt catcgtgcag cgcccgacca |
| | ccatgttcta cgtctacctg |
| 2281 | ggcatcgtgc tatccacgct gctcgtcatc gccgtgctgg |
| | aggtcaagtg ggccggggtc |
| 2341 | acagtcttcg agtggttcag gaacggccag ttctggatga |
| | cagcaagttg ctccgcctac |
| 2401 | ctcgccgccg tctgccaggt gctgaccaag gtgatattcc |
| | ggcgggacat ctccttcaag |
| 2461 | ctcacatcca agctaccctc gggagacgag aagaaggacc |
| | cctacgccga cctctacgtg |
| 2521 | gtgcgctgga cgccgctcat gattacaccc atcatcatca |
| | tcttcgtcaa catcatcgga |
| 2581 | tccgccgtgg ccttcgccaa ggttctcgac ggcgagtgga |
| | cgcactggct caaggtcgcc |
| 2641 | ggcggcgtct tcttcaactt ctgggtgctc ttccacctct |
| | accccttcgc caagggcatc |
| 2701 | ctggggaagc acggaaagac gccagtcgtg gtgctcgtct |
| | ggtgggcatt caccttcgtc |
| 2761 | atcaccgccg tgctctacat caacatcccc cacatgcata |
| | cctcgggagg caagcacaca |
| 2821 | acggtgcatg gtcaccatgg caagaagttg gtcgacacag |
| | ggctctatgg ctggctccat |
| 2881 | tgatgacttt gcccggacaa gacgacctga gacaagaaac |
| | aactcatcca ctcaacagtc |
| 2941 | agtgcatgca tccatctcat cgagaagcag agcccgccaa |
| | agtttgaatt ttttaatttt |
| 3001 | ttttcttcac ttttttgccc gtttcttttt agttttgtcc |
| | agaaaaaaga tggtgttgat |
| 3061 | ttgatttagt ttataattac ctgtggtaat taattatgta |
| | cttaattata cattccgcga |
| 3121 | acaacaaggg agacagacga cttacggggt actggctcgg |
| | gtggtaagag cttgcactgt |
| 3181 | actgtacatg ctcgacgatg tatagagatg cacagaggag |
| | aggatgggag tgctgggacc |
| 3241 | gtggggtgga cggcggtatt cttttagtat tatatatgga |
| | aacaataaat ttaatttcat |
| 3301 | taaaaaa |

A CslF6 amino acid sequence from corn (Zea mays) has about 82% sequence identity with the CslF6 from Brachypodium distachyon that has SEQ ID NO: 1. This corn CslF6 sequence is shown below with SEQ ID NO:9.

MAAVASAASSADMDRVAVAATEGQIGAVNDESWIAVDLSDDGLSADGA
DPGVALEDRPVFRTEKIKGVLLHPYRVLIFVRLIAFTLFVIWRISHRN
PDALWLWVTSIAGEFWFGFSWLLDQLPKLNPINRVPDLAALRQRFDRA
GGGAGGGTSLLPGLDVFVTTADPFKEPILSTANSVLSILAADYPVERN
TCYLSDDSGMLLTYEAMAEAAKFATVWVPFCRKHGIEPRGPESYFDLK
SHPYMGRSQEDFVNDRRRVRKDYDEFKARINGLDHDIKQRSDAYNAAR
GLKDGEPRATWMADGTQWEGTWVEPSENHRKGDHAGIVLVLLNHPSHS
RQLGPPASADNPLDLSMVDVRLPMLVYVSREKRPGHNHQKKAGAMNAL
TRCSAVLSNSPFILNLDCDHYINNSQALRAGICFMLGRDSDTVAFVQF
PQRFEGVDPTDLYANHNRIFFDGTLRALDGMQGPIYVGTGCLFRRITL
YGFDPPRINVGGPCFPALGGMFAKAKYEKPGLELTTKAAVAKGKHGFL
PMPKKSYGKSDAFADTIPMASHPSPFAAAAAAVVVADEATIAEAVAVC
AAAYEKKTGWGSDIGWVYGTVTEDVVTGYRMHIKGWRSRYCSIYPHAF
IGTAPINLTERLFQVLRWSTGSLEIFFSRNNPLFGSTFLHPLQRVAYI
NITTYPFTAIFLIFYTTVPALSFVTGHFIVQRPTTMFYVYLAIVLGTL
LILAVLEVKWAGVTVFEWFRNGQFWMTASCSAYLAAVCQVLVKVVFRR
DISFKLTSKQPAGDEKKDPYADLYVVRWTWLMVTPIIIILVNIIGSAV
AFAKVLDGEWTHWLKVAGGVFFNFWVLFHLYPFAKGILGRHGKTPVVV
LVWWAFTFVITAVLYINIPHIHGPGGKHGGAIGRHGGDAHHHGKKFDG
YYLWP

A nucleotide sequence that encodes the CslF6 protein from Zea mays with SEQ ID NO:9 is shown below as SEQ ID NO:10.

| | |
|---|---|
| 1 | cttccgccag gcaggcagag agggagggcg cgcccggcat tatggcggcg gtggcgtccg |
| 61 | ccgcgtcctc cgccgacatg gaccgcgtgg ccgtggccgc caccgagggc cagatcggcg |
| 121 | ccgtcaacga cgagagctgg atcgccgtcg acctcagcga cgacggcctg tccgccgacg |
| 181 | gcgccgaccc gggcgtcgcg ctcgaggacc gccccgtctt ccgcaccgag aagatcaagg |
| 241 | gcgtcctcct ccacccctac agggtgctga tcttcgtccg gctgatcgcg ttcacgctgt |
| 301 | tcgtgatctg gcgcatctcg caccgcaacc cggacgcgct gtggctgtgg gtgacgtcga |
| 361 | tcgcgggcga gttctggttc ggcttctcgt ggctgctgga ccagctgccg aagctgaacc |
| 421 | cgatcaaccg cgtgccggac ctggcggcgc tgcggcagcg gttcgaccgc gcgggcgggg |
| 481 | gcgccggcgg agggacgtcg ctgctgccgg ggctggacgt gttcgtgacg acggcggacc |
| 541 | cgttcaagga gccgatactg agcacggcca actccgtgct gtccatcctg gccgccgact |
| 601 | accccgtgga gcgcaacacg tgctacctct ccgacgactc cgggatgctg ctcacctacg |
| 661 | aggccatggc ggaggccgcc aagttcgcca ccgtctgggt gcccttctgc cgcaagcacg |
| 721 | gcatcgagcc gcgcggcccc gagagctact tcgacctcaa gtcccaccct tacatgggcc |
| 781 | gctcccagga ggacttcgtc aacgaccgcc gccgcgtgcg caaggactac gacgagttca |
| 841 | aggcgcgcat caacggcctc gaccacgaca tcaagcagag gtccgacgcg tacaacgccg |
| 901 | ccagggggct caaggacggc gagcccaggg caacctggat ggccgacggc acccagtggg |
| 961 | agggcacctg ggtcgagccc tccgagaacc accgcaaggg agaccacgcc ggcatcgtac |
| 1021 | tggtgcttct gaaccacccg agccacagcc gtcagctcgg cccgccggcg agcgctgaca |
| 1081 | acccgctgga cttgagcatg gtggacgtgc ggctcccgat gctggtgtac gtgtcccgcg |
| 1141 | agaagcggcc cggtcacaac caccagaaga aggccggcgc catgaacgcg ctgacccggt |
| 1201 | gctccgcggt gctgtccaac tcgcccttca tcctcaacct ggactgcgac cactacatca |
| 1261 | acaactcgca ggcgctgcgc gcgggcatct gcttcatgct cggccgcgac agcgacacgg |
| 1321 | tggccttcgt gcagttcccg cagcgcttcg agggcgtgga ccccacggac ctgtacgcca |
| 1381 | accacaaccg catcttcttc gacggcacgc tcagggcgct ggacggcatg cagggcccca |
| 1441 | tctacgtggg cacgggctgc ctgttccgcc gcatcacgct ctacggcttc gacccgccgc |
| 1501 | ggatcaacgt gggcgggccg tgcttcccgg cgctgggcgg catgttcgcc aaggccaagt |
| 1561 | acgagaagcc cggactggag ctcaccacca aggccgccgt ggccaagggc aagcacggct |
| 1621 | tcctgcccat gcctaagaag tcgtacggca agtcggacgc gttcgcggac accatcccga |
| 1681 | tggcgtcgca cccgtcgccg ttcgcggcgg ccgccgccgc cgtcgtcgtg gcggacgagg |
| 1741 | cgaccatcgc cgaggcagtg gcggtgtgcg cggcggcgta cgagaagaag accgggtggg |
| 1801 | gcagcgacat cggatgggtg tacggcacgg tgacggagga cgtggtgacg gggtaccgga |
| 1861 | tgcacatcaa ggggtggcgg tcccgctact gctccatcta cccgcacgcc ttcatcggca |
| 1921 | ccgcccccat caacctgacg gagcggctgt tccaggtgct ccgctggtcc acgggttccc |
| 1981 | tggagatctt cttctcccgg aacaacccgc tgttcgggag cacgttcctg cacccgctgc |
| 2041 | agcgcgtggc gtacatcaac atcacgacgt acccgttcac ggccatcttc ctcatcttct |
| 2101 | acacgacggt gccggcgctg tcgttcgtga cggggcactt catcgtgcag cggcccacca |
| 2161 | ccatgttcta cgtgtacctg gccatcgtgc tgggcacgct gctgatcctg gccgtcctgg |
| 2221 | aggtgaagtg ggcgggcgtg acggtgttcg agtggttcag gaacgggcag ttctggatga |
| 2281 | cggccagctg ctccgcgtac ctggccgcgg tgtgccaggt gctggtgaag gtggtgttca |
| 2341 | ggcgggacat ctcgttcaag ctgacgtcga agcagcccgc gggggacgag aagaaggacc |
| 2401 | cctacgcaga cctgtacgtg gtgcgctgga cctggctcat ggtgacgccc atcatcatca |
| 2461 | tcctcgtcaa catcatcggc tccgccgtgg ccttcgccaa ggtgctggac ggcgagtgga |
| 2521 | cgcactggct caaggtggcc ggcggcgtct tcttcaactt ctgggtgctc ttccacctct |
| 2581 | accctttcgc caagggcatc ctggggaggc acggcaagac ccccgtggtg gtgctcgtct |
| 2641 | ggtgggcctt caccttcgtc atcaccgccg tgctgtacat caacatccca cacatccacg |
| 2701 | ggcccggcgg caagcacggc ggcgcgatcg gaaggcacgg cggcgacgca caccaccatg |
| 2761 | gcaagaagtt cgacggctac tacctctggc cgtga |

A CslF6 amino acid sequence from Sorghum (Sorghum bicolor) has about 82% sequence identity with the CslF6 from Brachypodium distachyon that has SEQ ID NO:1. This Sorghum CslF6 sequence is shown below with SEQ ID NO: 11.

| | |
|---|---|
| 1 | MAPGGGDGRR NGEGQQQANG NNNNNNSNAK AKHGCVCGFP |
| 41 | VCACAGAAAV ASAASSADMD RVAAAQTEGQ IGAVNDESWI |
| 81 | AVDLSDDLSG DGGGADPGVA IEDRPVFRTE KIKGILLHPY |
| 121 | RVLIFVRLIA FTLFVIWRIS HRNPDAMWLW VTSIAGEFWF |
| 161 | GFSWLLDQLP KLNPINRVPD LAVLRQRFDR ADGTSRLPGL |
| 201 | DIFVTTADPF KEPILSTANS ILSILAADYP VERNTCYLSD |
| 241 | DSGMLLTYEA MAEAAKFATV WVPFCRKHGI EPRGPESYFE |
| 281 | LKSHPYMGRS QEDFVNDRRR VRKEYDEFKA RINGLEHDIK |
| 321 | QRSDAFNAAR GLKDGEPRAT WMADGNQWEG TWVEPSENHR |
| 361 | KGDHAGIVYV LLNHPSHSRQ LGPPASADNP LDFSMVDVRL |
| 401 | PMLVYVSREK RPGFNHEKKA GAMNALTRCS AVISNSPFIL |
| 441 | NLDCDHYINN SQALRAGICF MLGRDSDTVA FVQFPQRFEG |
| 481 | VDPTDLYANH NRIFFDGTLR ALDGMQGPIY VGTGCMFRRI |
| 521 | TLYGFDPPRI NVGGPCFPSL GGMFAKTKYE KPGLELTTKA |
| 561 | AVAKGKHGFL PLPKKSYGKS DAFVDTIPRA SHPSPFLSAD |
| 601 | EAAAIVADEA MITEAVEVCT AAYEKKTGWG SDIGWVYGTV |
| 641 | TEDVVTGYRM HIKGWRSRYC SIYPHAFIGT APINLTERLY |
| 681 | QVLRWSTGSL EIFFSRNNPL FGSTFLHPLQ RVAYINITTY |
| 721 | PFTALFLIFY TTVPALSFVT GHFIVQRPTT MFYVYLAIVL |
| 761 | GTLLILAVLE VKWAGVTVFE WFRNGQFWMT ASCSAYLAAV |
| 801 | CQVLVKVVFR RDISFKLTSK QPAGDEKKDP YADLYVVRWT |
| 841 | WLMVTPIIII LVNIIGSAVA FAKVLDGEWT HWLKVAGGVF |
| 881 | FNFWVLFHLY PFAKGLLGRH GKTPVVVLVW WAFTFVITAV |
| 921 | LYINIPHIHG PGGKHGGAIG KHGAAHHGKK FDLDNLSYNW |
| 961 | P |

A nucleotide sequence that encodes the CslF6 protein from Sorghum bicolor with SEQ ID NO:11 is shown below as SEQ ID NO:12.

| | |
|---|---|
| 1 | gagggcgcag ccggcattat ggcgccgggc ggcggagacg gccggcgcaa cggcgaggga |
| 61 | cagcagcagg cgaacggcaa caacaacaac aacaacagca acgctaaggc taagcacggg |
| 121 | tgcgtgtgcg ggttccccgt gtgcgcgtgc gccggcgcgg ccgcggtggc gtccgcggcc |
| 181 | tcctccgccg acatggaccg cgtggccgcc gcgcagaccg agggccagat cggcgccgtc |
| 241 | aacgacgaga gctggatcgc cgtcgacctg agcgacgacc tctccggcga cggcggcggc |
| 301 | gccgaccccg gcgtcgcgat cgaggaccgc cccgtcttcc gcaccgagaa gatcaagggc |
| 361 | atcctcctcc acccctacag ggtgctcatc ttcgtgcgcc tgatcgcgtt cacgctgttc |
| 421 | gtcatctggc gtatctcgca ccgcaacccg gacgcgatgt ggctgtgggt gacgtcgatc |
| 481 | gcgggcgagt tctggttcgg cttctcctgg ctgctggacc agctccccaa gctgaacccg |
| 541 | atcaaccgcg tcccggacct cgcggtgctc cggcagcggt tcgaccgcgc cgacggcacg |
| 601 | tcccgcctcc cgggcctgga catcttcgtc accacggcgg acccgttcaa ggagcccatc |
| 661 | ctgagcacgg ccaactccat cctctccatc cttgccgccg actaccccgt ggagcgcaac |
| 721 | acgtgctacc tctccgacga ctccgggatg ctgctcacct acgaggccat ggcggaggcc |
| 781 | gccaagttcg ccaccgtctg ggtgcccttc tgccggaagc acggcatcga gcctcgtggc |
| 841 | cccgagagct acttcgagct caagtcgcac ccctacatgg ggaggtcgca ggaggacttc |
| 901 | gtcaacgacc gccgccgtgt ccgcaaggag tacgacgagt tcaaggcccg gatcaatggc |
| 961 | ctcgagcatg atatcaagca gaggtccgac gcgtttaacg ccgctagggg gcttaaggac |
| 1021 | ggcgagccca gagctacgtg gatggccgac gggaaccagt gggagggcac atgggttgag |
| 1081 | ccatcggaga accaccgcaa gggtgaccac gccggcatcg tctatgtgct tctgaaccac |
| 1141 | ccgagccaca gccgtcagct cggcccgccg gcgagcgcgg acaacccgct ggacttcagc |
| 1201 | atggtggacg ttcgcctccc catgctggtg tacgtctccc gtgagaagcg gcccgggttc |
| 1261 | aaccacgaga agaaggccgg cgccatgaac gcgctgaccc gctgctccgc cgtgatctcc |
| 1321 | aactcgccct tcatcctcaa cctggactgc gaccactaca tcaacaactc gcaggcgctt |
| 1381 | cgcgccggca tctgcttcat gctcggccgg gacagcgaca cggtggcgtt cgtgcagttc |
| 1441 | ccgcagcggt tcgagggcgt ggaccccacg gacctgtacg ccaaccacaa ccgcatcttc |
| 1501 | ttcgacggca cgctccgggc gctggacggc atgcagggcc ccatctacgt cggcaccggc |
| 1561 | tgcatgttcc gccgcatcac gctctacggc ttcgacccgc cgaggatcaa cgtcggcggg |
| 1621 | ccgtgcttcc cgtcgctcgg cggcatgttc gccaagacca agtacgagaa gcccgggctg |
| 1681 | gagctcacca ccaaggccgc cgtcgccaag ggcaagcacg gcttcctccc gttgcccaag |
| 1741 | aagtcgtacg gcaagtcgga cgcgttcgtc gacaccatcc cgagggcgtc tcacccgtcg |
| 1801 | ccgttcctga gcgccgacga ggccgccgcc atcgtcgccg acgaggccat gatcaccgag |
| 1861 | gccgtggagg tgtgcacggc ggcgtacgag aagaagaccg gctggggcag cgacatcggc |
| 1921 | tgggtgtacg gcaccgtcac cgaggacgtg gtgacggggt accggatgca catcaagggg |
| 1981 | tggcggtctc gctactgctc catctacccg cacgccttca tcggcaccgc cccgatcaac |
| 2041 | ctgacggagc ggctgtacca ggtgctccgc tggtccacgg ggtcgctgga gatcttcttc |
| 2101 | tcccggaaca acccgctgtt cggcagcacg ttcctgcacc cgctgcagcg cgtggcgtac |
| 2161 | atcaacatca ccacctaccc gttcacggcg ctgttcctca tcttctacac caccgtgccg |
| 2221 | gcgctgtcgt tcgtgacggg gcacttcatc gtgcagcggc cgaccaccat gttctacgtg |
| 2281 | tacctggcca tcgtgctggg gacgctgctc atcctggccg tcctggaggt gaaatgggcg |
| 2341 | ggcgtcaccg tcttcgagtg gttcaggaac gggcagttct ggatgacggc cagctgctcc |
| 2401 | gcgtacctgg ccgccgtgtg ccaggtgctg gtgaaagtgg tgttccggcg agacatctcc |
| 2461 | ttcaagctca catccaagca gcccgccggc gacgagaaga aggaccccta cgccgacctg |
| 2521 | tacgtggtgc gctggacctg gctcatggtg acccccatca tcatcatcct cgtcaacatc |
| 2581 | atcggatccg ccgtggcgtt cgccaaggtg ctggacggcg agtggacgca ctggctcaag |
| 2641 | gtggccggcg gcgtcttctt caacttctgg gtgctgttcc acctctaccc gttcgccaag |
| 2701 | ggcctcctcg ggaggcacgg caagaccccc gtggtggtgc tcgtctggtg ggcattcacc |
| 2761 | ttcgtcatca ccgccgtgct ctacatcaac atcccccaca tccatggccc cggcggcaag |
| 2821 | cacggcggcg cgatcggaaa gcacggcgcc gcccaccacg gcaagaagtt cgacctcgac |
| 2881 | aacctctcct acaactggcc gtgaag |

### Overexpression of CslF6

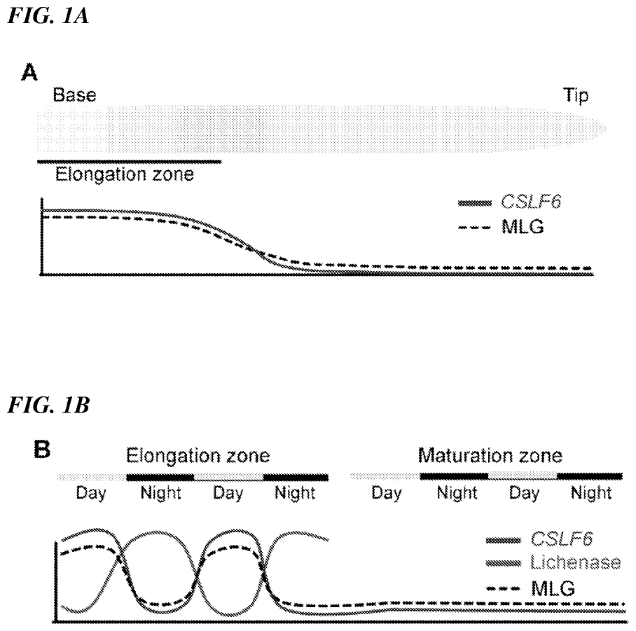

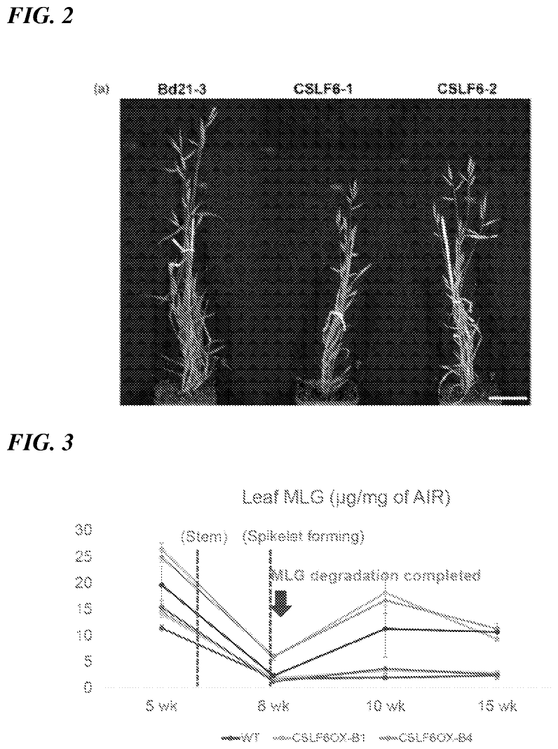

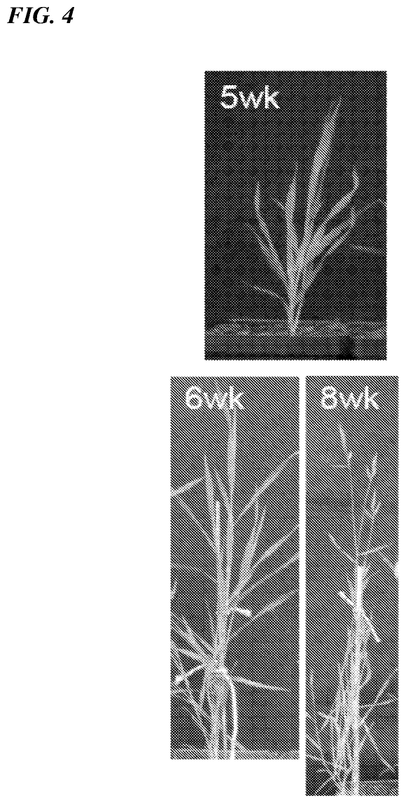

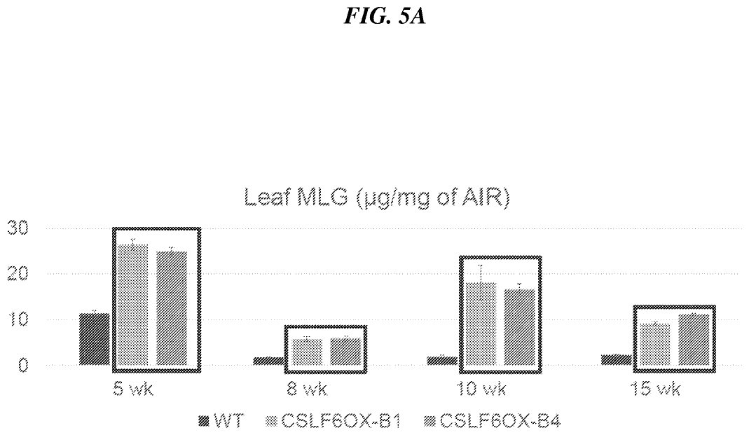

To increase the levels of MLG in Sorghum bicolor, methods of increasing MLG synthesis and reducing or preventing MLG degradation in the plant are described. In plants, MLG abundance is controlled by MLG synthase, CslF6 and MLG hydrolase, lichenase through developmental program (FIGS. 1A-B). Overproduction of MLG hampers growth in some species, such as Brachypodium (FIG. 2), rice and barley. Nevertheless, transgenic Sorghum was produced to overexpress an MLG synthase (CslF6) from either Brachypodium or Sorghum (constructs named BdCslF6 and SbCslF6, respectively), which generate a different frequency of β1-3 linkages in MLG, resulting in different types of the fine structure of this polymer.

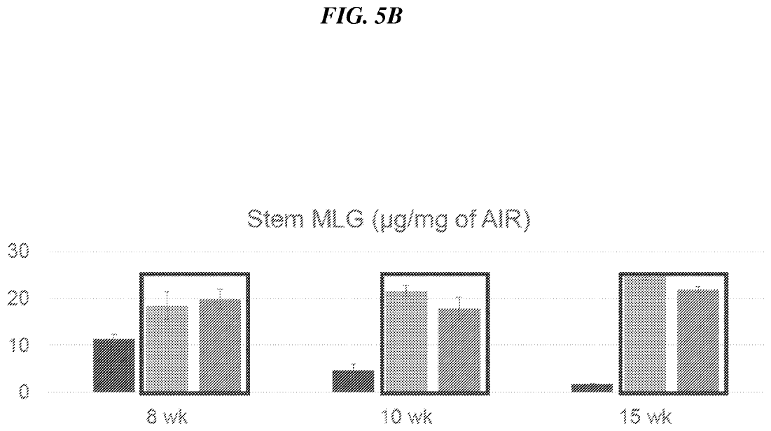

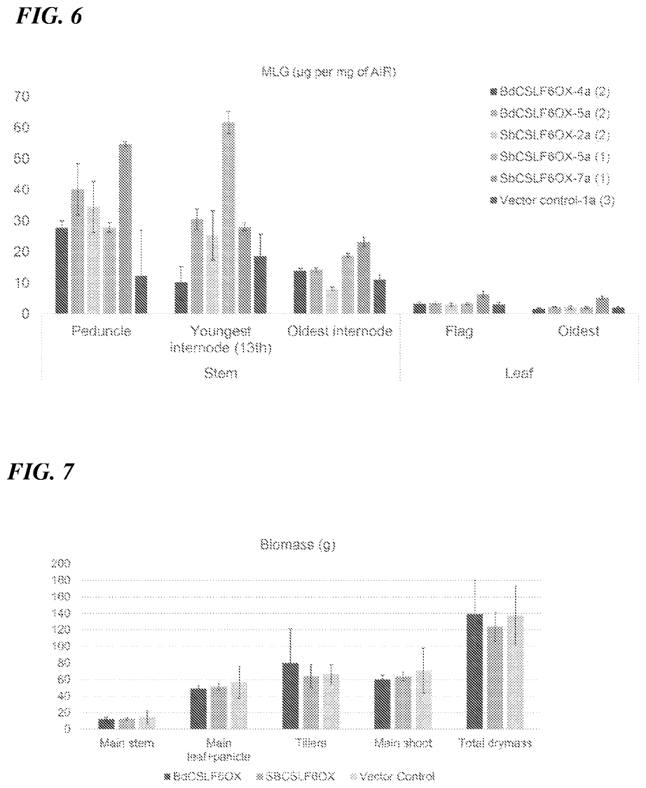

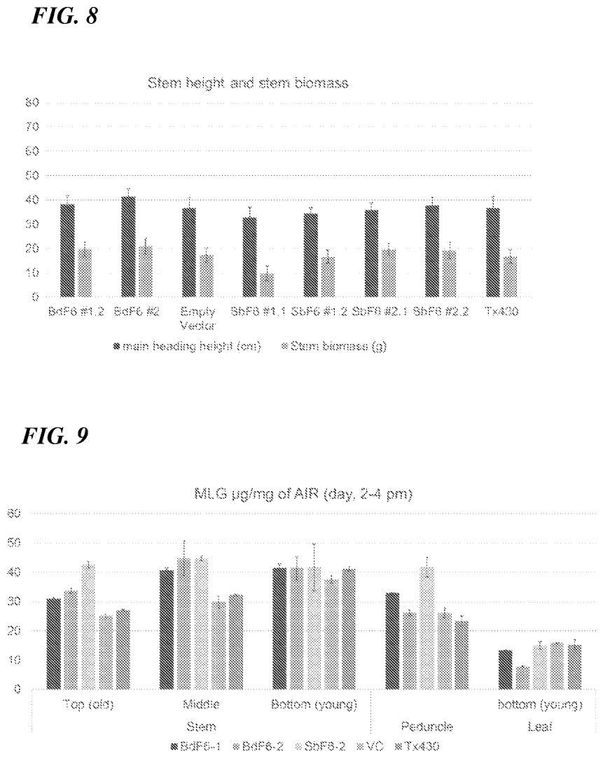

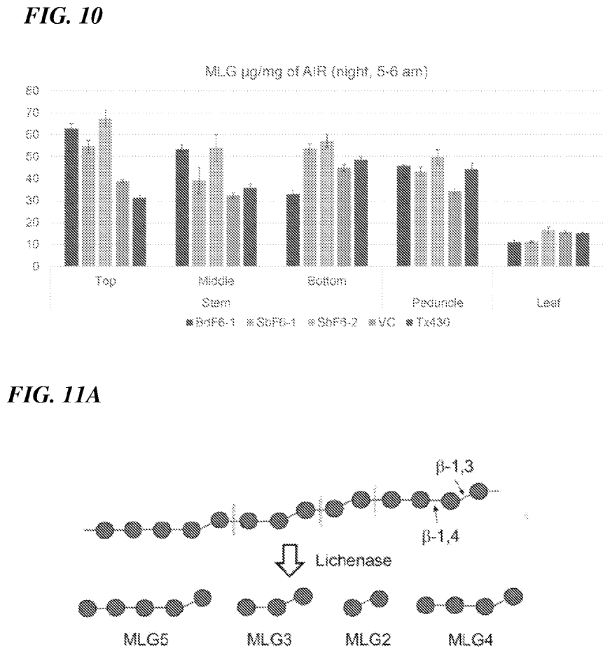

The transgenic Sorghum grown in a greenhouse showed higher MLG levels compared to the vector control without any noticeable growth defects (FIGS. 6 and 7). Field trials in East Lansing, MI were conducted with the T3 generation of CslF6-sorghum transgenics and found no defects in growth (FIG. 8). MLG analyses from the field-grown Sorghum were performed during development within single stem internodes. The transgenic lines had higher levels of MLG compared to wild-type and vector controls, even in the fully elongated region of the internode where MLG degradation normally occurs (FIGS. 9-10). MLG abundance was determined in Sorghum young leaves (FIG. 13). High MLG and low starch synthesis was found in the actively elongating region of leaves. On the contrary, unchanged low level of MLG and dynamic starch metabolism were observed in the mature region of leaves.

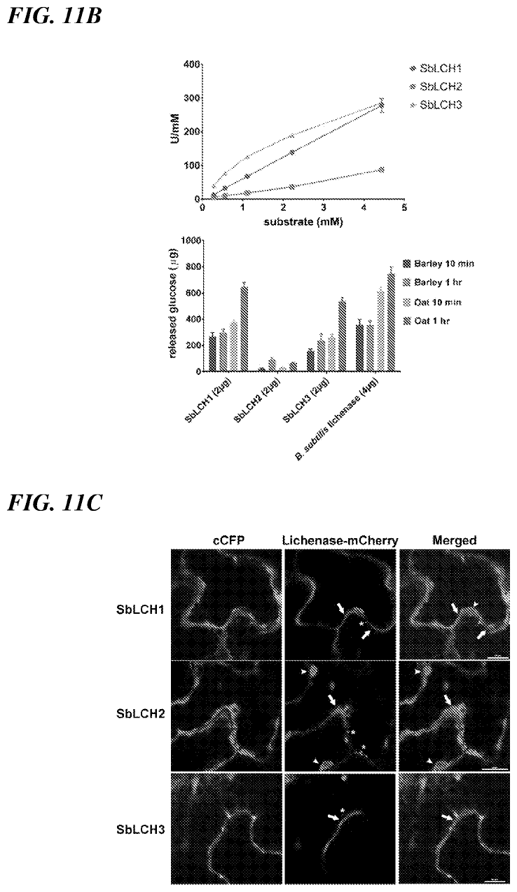

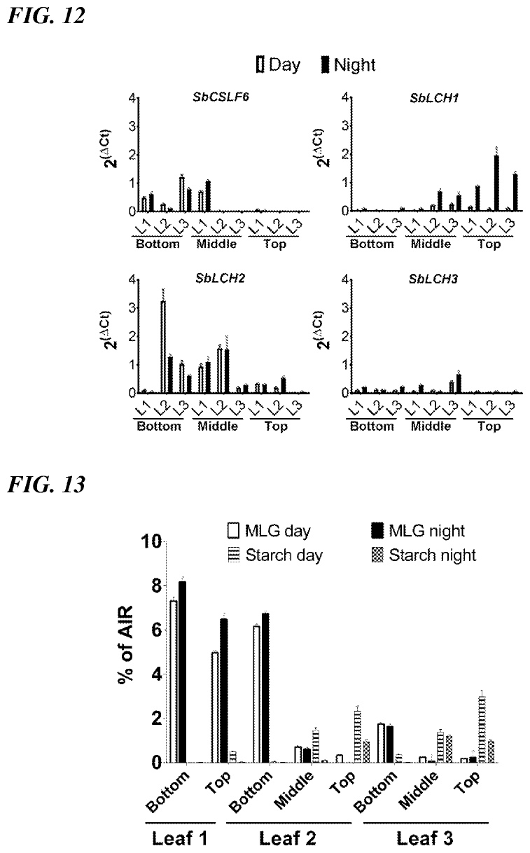

Furthermore, to modulate and reduce MLG degradation in Sorghum and increase levels of MLG, three Sorghum MLG endoglucanases (also called lichenases) were identified and characterized (FIGS. 11A-C). The three lichenases have similar enzymatic activity, with an optimal pH near 5, between 25° C. and 45° C. and digest both synthetic substrate and MLG in flours (FIG. 11B). The three enzymes were localized in the apoplast, where lichenase activity is expected to take place (FIG. 11C). The lichenases' expression patterns were determined during development and light conditions, which led us to identify the lichenase that is responsible for MLG degradation in sorghum (FIG. 12).

### Vacuolar Targeting Polypeptide

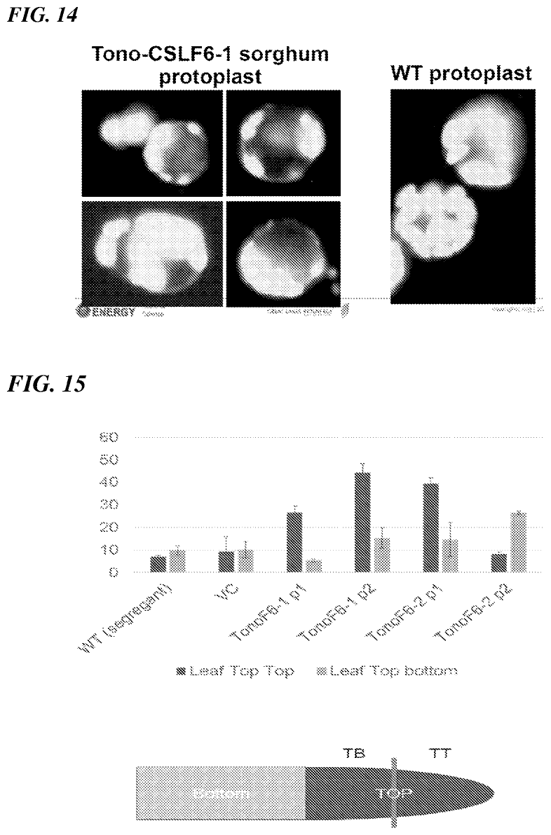

A variety of vacuolar targeting polypeptides can be linked to a Csl protein, such as CslF6, to form the fusion protein that can be targeted to the tonoplast (FIG. 14). Expression of the fusion protein can result in higher levels of MLG production in plants such as Sorghum compared to wild type plants not expressing the fusion protein (FIG. 15). Vacuoles perform a multitude of functions in plant cells, including the storage of amino acids and sugars. Vacuoles are the largest plant organelles, constituting up to 90% of the cell volume. They are indispensable for multiple cellular functions, including the maintenance of turgor to shape the individual cell and the entire plant. Two types of vacuoles have been described in plants, a lytic vacuole and a protein storage vacuole. The lytic vacuole has acidic pH, is abundant in vegetative tissues, and is homologous to the animal lysosome. The protein storage vacuole (PSV) has a neutral pH and is the main protein storage compartment in developing seeds.

Tonoplast-localized transporters catalyze the import and release of these molecules. For example, the vacuolar targeting polypeptide can be a beta fructosidase 4 (βFruct4) polypeptide vacuole targeting domain. Fruct4 genes encode vacuolar forms of invertase. Fruct4 proteins comprise an N-terminal cytosolic domain that can transport the protein from the ER to the central vacuole (the “vacuole targeting domain”).

Fruct4 protein has a short cytosolic region of 42 amino acids at the N-terminus (“signal”) followed by a single transmembrane domain (“TMD”) and a luminal C-terminal region, which contains a functional fructosidase domain. The AtFruct4 the N-terminal region contains the sequence information for trafficking to the tonoplast (the “vacuole targeting domain”). Jung, et. al., Identification of Sorting Motifs of AtβFruct4 for Trafficking from the ER to the Vacuole Through the Golgi and PVC, Traffic (2011) 12(12): 1774-1792.

An amino acid sequence for the Fruct4 vacuole targeting domain from Arabidopsis thaliana is shown below with SEQ ID NO: 13.

MASSDALLPI SAREEEPLCP YTRLPMADPN QETHGPRRRR
PFKG

A nucleotide sequence that encodes the Fruct4 vacuole targeting domain from Arabidopsis thaliana with SEQ ID NO:13 is shown below as SEQ ID NO:14.

| | |
|---|---|
| 1 | cgatgctctc ttgccaatct ccgccagaga agaagaacca ttatgtcctt acacgagatt |
| 61 | accaatggcc gacccgaatc aagaaaccca tggcccccgg agaagaagac cctttaaagg |
| 121 | tctcctcgcc gtctcatttg gt |

In some cases, another vacuole targeting domain can include a tonoplast intrinsic protein (TIP), including TIP1-1 or TIP3-1. TIPs facilitate the membrane transport of water and other small molecules across the plant vacuolar membrane. In barley, pea and Arabidopsis root tips, the lytic vacuole is marked by the presence of TIP1-1. TIP3-1 localizes to the protein storage vacuole. Delivery of TIP proteins to the tonoplast is thought to occur by vesicle-mediated traffic from the endoplasmic reticulum to the vacuole, and at least two pathways have been proposed, one that is Golgi-dependent and another that is Golgi-independent.

The TIP1-1 and TIP3-1 whole protein can be fused to a Csl protein, such as Csl6, to form a fusion protein that is targeted to the tonoplast (FIG. 14). An amino acid sequence for TIP1-1 from Arabidopsis thaliana is shown below with SEQ ID NO: 15.

MPIRNIAIGR PDEATRPDAL KAALAEFIST LIFVVAGSGS
GMAFNKLTEN GATTPSGLVA AAVAHAFGLF VAVSVGANIS
GGHVNPAVTF GAFIGGNITL LRGILYWIAQ LLGSVVACLI
LKFATGGLAV PAFGLSAGVG VLNAFVFEIV MTFGLVYTVY
ATAIDPKNGS LGTIAPIAIG FIVGANILAG GAFSGASMNP
AVAFGPAVVS WTWTNHWVYW AGPLVGGGIA GLIYEVFFIN
TTHEQLPTTD Y

A nucleotide sequence that encodes the TIP1-1 from Arabidopsis thaliana with SEQ ID NO: 15 is shown below as SEQ ID NO:16.

| | |
|---|---|
| 1 | gctcttctag catcttgaag cttaaatctc agccgtccga tcatgccgat cagaaacatc |
| 61 | gccattggcc gtccagatga agccacccgt cccgatgcct taaaggcggc gttggctgag |
| 121 | ttcatttcaa ctttgatctt tgtcgtcgcc ggttcaggct ctggcatggc tttcaacaag |
| 181 | ctcactgaaa acggagccac cactccttct ggtctcgtag ctgctgcagt ggctcatgcc |
| 241 | tttggactct tcgtcgctgt ctcagttggt gccaacatct ctggtggaca cgttaaccct |
| 301 | gccgtcactt tcggtgcttt cattggtggt aacatcactc tcctccgtgg tatcctctac |
| 361 | tggattgctc agcttctcgg ctccgtcgtc gcttgcctca tccttaaatt cgccaccgga |
| 421 | ggcttggctg tgcccccttt tggtctctct gctggagtag gagtgttgaa cgctttcgtt |
| 481 | ttcgagatcg tgatgacatt cgggcttgtt tacaccgtct acgctacagc cattgacccc |
| 541 | aaaaacggga gtcttggaac aattgctccc atcgcaatcg gtttcattgt tggagccaac |
| 601 | atcttagctg gaggagcttt ctctggagcc tccatgaatc ccgccgtggc tttcggacca |
| 661 | gcggtggtga gctggacatg gaccaaccac tgggtctact gggccggacc tctcgtcggc |
| 721 | ggtggaatcg ctggactcat ctacgaagtt ttcttcatca acaccacaca cacgagcagc |
| 781 | tccaaccaca gactactgaa ttaatctctc tttttctctc ttgtgtaatt ttattcgact |
| 841 | ttgaatttga attttaatgt ctttttaatt tccgtttgtg taatttcgaa tcatcaaggg |
| 901 | gtttatgatc gttcgatctt ttagatgaat ccttgtccgt tggtttcaag agtggccatt |
| 961 | gcaatttcga ctatataatg tgaattatgt atc |

An amino acid sequence for TIP3-1 from Arabidopsis thaliana is shown below with SEQ ID NO: 17.

MATSARRAYG FGRADEATHP DSIRATLAEF LSTFVFVFAA
EGSILSLDKL YWEHAAHAGT NTPGGLILVA LAHAFALFAA
VSAAINVSGG HVNPAVTFGA LVGGRVTAIR AIYYWIAQLL
GAILACLLLR LTTNGMRPVG FRLASGVGAV NGLVLEIILT
FGLVYVVYST LIDPKRGSLG IIAPLAIGLI VGANILVGGP
FSGASMNPAR AFGPALVGWR WHDHWIYWVG PFIGSALAAL
IYEYMVIPTE PPTHHAHGVH QPLAPEDY

A nucleotide sequence that encodes the TIP3-1 from Arabidopsis thaliana with SEQ ID NO: 17 is shown below as SEQ ID NO:18.

| | |
|---|---|
| 1 | acactttgtt gttaatttca agtgtttgat cataatggca acatcagctc gtagagcata |
| 61 | cggtttcggt agagccgatg aggctacaca ccctgactcc attagagcaa ctttagctga |
| 121 | gtttctctcc acttttgtct tcgtctttgc agctgaaggc tctatcctct ctctcgataa |
| 181 | gttgtattgg gaacatgcgg ctcatgcggg gacaaacaca ccaggagggc tgattttagt |
| 241 | agcgttggct catgcgtttg ctctgtttgc tgctgtttca gcagccatta atgtctccgg |
| 301 | cggacacgtt aacccggcag tcacttttgg tgctcttgtt ggaggcagag ttacagcgat |
| 361 | ccgcgccatc tactactgga tcgctcagct tcttggagcc atcctcgctt gtctcttgtt |
| 421 | aaggctcaca acaaacggca tgagaccagt tggtttccgt ctagcatcag gtgttggagc |
| 481 | ggttaatgga cttgtattag agatcatttt aacatttggc ttagtctacg tagtgtattc |
| 541 | cactttgatt gatccaaaac gtggaagcct cgggatcata gcaccgcttg caatcggact |
| 601 | catagttggg gcaaacatct tagtaggtgg accattttct ggtgcttcga tgaatccagc |
| 661 | tagagctttt ggtccagcgt tggtgggatg gagatggcat gaccactgga tctattgggt |
| 721 | cggaccattc atcggtagtg ctttagccgc ccttatatat gagtacatgg tcatacccac |
| 781 | cgaaccacct acccaccacg cacatggtgt acaccagccc ttggcccctg aagattacta |
| 841 | gatggaactt tcctcatgtc accactgctt ttgttcgttg ttcaagctct ctttgtctgt |
| 901 | atgatgagat cacttgcata gatacttttt ttcttatgct actcttatgt tgtaataata |
| 961 | aaaaataaag aacgatttcg ctaaaaaaaa aaaaaaaa |

An amino acid sequence for a TIP protein from Brachypodium distachyon is shown below with SEQ ID NO: 19.

MPVSRIAIGS PGELSHPDTF KAAVAEFISM LIFVFAGSGS
GMAFGKLTDG GATTPSGLIA AALAHAFALF VAVSVGANIS
GGHVNPAVTF GAFIGGNISL LKAIVYWVAQ LLGSTAACLL
LQISTGGASV GAFSLSAGVG VWNAVVFEIV MTFGLVYTVY
ATAVDPKRGD LGVIAPIAIG FIVGANILAG GAFDGASMNP
AVSFGPAVVS GVWENHWVYW LGPFAGAAIA ALVYDIVFIG
QRPHDQLPSA EY

An amino acid sequence for another TIP protein from Brachypodium distachyon is shown below with SEQ ID NO: 20.

MPISRVAVGS HHEVYQAGAL KAAVAEFIST LIFVFAGQGS
GMAFNKLNAG GAATPAGLIS AAIAHAFALF VAVSVGANIS
GGHVNPAVTF GAFLGGNITL FRGLLYWVAQ LLGSTAACFL
LRFSTGGLPT GTFGLTGVSV WEALVLEIVM TFGLVYTVYA
TAVDPKKGSI GTIAPLAIGF IVGANILVGG AFDGASMNPA
VSFGPALVSW EWGYQWVYWV GPLIGGGLAG VIYEVLFISH
THEQLPTTDY

An amino acid sequence for a TIP1-1 protein from Triticum aestivum is shown below with SEQ ID NO: 21.

EFISTLIFVF AGQGSGMAFS KLSPDGAATP AGLISAAIAH
AFALFVAVSV GANISGGHVN PAVTFGAFVG GNITLFRGLL
YWIAQLLGST AACFLLRFST GGLPTGTFGL SGIGAWEAVV
LEIVMTFGLV Y

A nucleotide sequence that encodes the TIP1-1 protein from Triticum aestivum with SEQ ID NO:21 is shown below as SEQ ID NO:22.

| | |
|---|---|
| 1 | acacgagccg atccatccat ccagaaaaaa aaaaaagagt gcccgtcgaa aatgccggtg |
| 61 | cgcaggatcg ccatcggcgc cccgggggag ctgtcccacc cggacacctt ccgcgccggc |
| 121 | gtcgccgagt tcatctccat gctcatcttc gtcttcgccg gctcaggctc cggcatggcc |
| 181 | ttcggcaagc tgactgacgg cggcgcgacg acgccggcgg ggcttatcgc ggcggcgctg |
| 241 | gcgcacgcgt tcgcgctctt cgtggccgtc tccgtgggcg ccaacatctc cggcgggcac |
| 301 | gtgaacccgg ccgtcacatt cggcgccttc gtgggcggca acatcagcct gctcaaggcc |
| 361 | gtggtgtact gggtggcgca gctgctcggc tccgtcgtgg cctgcctcct cctcaagatc |
| 421 | gccaccggcg gcgaggccgt gggcgccttc tcgctctccg ccggcgtcgg cgtctggaac |
| 481 | gccgtggtgt tcgagatcgt catgaccttc gggctcgtct acacggtgta cgccaccgcg |
| 541 | gtcgacccca agcgcggcga cctcggggtg atcgcgccca tcgccatcgg cttcatcgtc |
| 601 | ggcgccaaca tcctcgccgg cggcgccttc gacggcgcct ccatgaaccc cgccgtctcc |
| 661 | ttcggccccg ctgtcgtcag cggcgtctgg gagaaccact gggtctactg gctcggcccc |
| 721 | ttcgtcggcg ccgccatcgc cgccctcgtc tacgacatct gcttcatcgg ccagcgcccc |
| 781 | cacgagcagc tccccaccgc cgactactga aaacctcaat ctcatctcgt ccatccgtca |
| 841 | tcgccatgag agattgaaat cgcttcaaaa attccagcaa gtgtacgtcg tcgcgtcatc |
| 901 | atcagtaagc cagtttagtt tcattgcgct atcttgtcct tgagaaattc agggtaaacg |
| 961 | gtgtgtgcga gccagtcagt caggaataat ctcatccttc tcttatcttg gtgtatgtat |
| 1021 | gtacgtcctc aattatgaat gcttctattc catggtcaag attgatgttt gtacaaaaaa |
| 1081 | aaaaaaaaaa aa |

An amino acid sequence for a TIP1-1 protein from Zea mays is shown below with SEQ ID NO: 23.

MPINRIALG SHQEVYHPG ALKAAFAEF ISTLIFVFA
GQGSGMAFS KLTGGGPTT PAGLIAAAV AHAFALFVA
VSVGANISG GHVNPAVTF GAFVGGNIT LFRGLLYWV
AQLLGSTVA CFLLRFSTG GQATGTFGL TGVSVWEAL
VLEIVMTFG LVYTVYATA VDPKKGSLG TIAPIAIGF
IVGANILVG GAFDGASMN PAVSFGPAL VSWEWGYQW
VYWVGPLIG GGLAGVIYE LLFISHTHE QLPSTDY

The nucleic acids and polypeptides allow identification and isolation of related nucleic acids and their encoded enzymes that provide a means for production of healthy plants with increased glucan.

The related nucleic acids can be isolated and identified by mutation of the SEQ ID NOS: 2, 3, 6, 8, 10, 12, 14, 16, 18, and 22 nucleic acid sequences and/or by hybridization to DNA and/or RNA isolated from other plant species using segments of these nucleic acids as probes. The sequence of the CSLF6, Fruct4, Tip1-1, and Tip3-1 proteins (e.g., SEQ ID NOs: 1, 5, 7, 9, 11, 13, 15, 17, 19, 20, 21, and 23) can also be examined and used a basis for designing alternative CslF6, Fruct4, Tip1-1, and/or Tip3-1 nucleic acids that encode related CslF6, Fruct4, Tip1-1, and/or Tip3-1 polypeptides.

The CslF6, Fruct4, Tip1-1, and/or Tip3-1 nucleic acids described herein can include any nucleic acid that can selectively hybridize to any of SEQ ID NOS: 2, 3, 6, 8, 10, 12, 14, 16, 18, or 22 nucleic acids. The term “selectively hybridize” includes hybridization, under stringent hybridization conditions, of a nucleic acid sequence to a specified nucleic acid target sequence (e.g., any of the SEQ ID NOS: 2, 3, 6, 8, 10, 12, 14, 16, 18, or 22 nucleic acids) to a detectably greater degree (e.g., at least 2-fold over background) than its hybridization to non-target nucleic acid sequences. Such selective hybridization substantially excludes non-target nucleic acids. Selectively hybridizing sequences typically have about at least 40% sequence identity, or at least 50% sequence identity, or at least 60% sequence identity, or at least 70% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity, or 100% sequence identity (or complementarity) with each other. In some embodiments, a selectively hybridizing sequence has about at least about 80% sequence identity or complementarity with SEQ ID NOS: 2, 3, 6, 8, 10, 12, 14, 16, 18, or 22.

Thus, the nucleic acids of the invention include those with about 500 of the same nucleotides as SEQ ID NOS: 2, 3, 6, 8, 10, 12, 14, 16, 18, or 22, or about 600 of the same nucleotides, or about 700 of the same nucleotides, or about 800 of the same nucleotides, or about 900 of the same nucleotides, or about 1000 of the same nucleotides, or about 1100 of the same nucleotides, or about 1200 of the same nucleotides as SEQ ID NOS: 2, 3, 6, 8, 10, 12, 14, 16, 18, or 22. The identical nucleotides or amino acids can be distributed throughout the nucleic acid or the protein, and need not be contiguous.

Note that if a value of a variable that is necessarily an integer, e.g., the number of nucleotides or amino acids in a nucleic acid or protein, is described as a range, e.g., 90-99% sequence identity what is meant is that the value can be any integer between 90 and 99 inclusive, i.e., 90, 91, 92, 93, 94, 95, 96, 97, 98 or 99, or any range between 90 and 99 inclusive, e.g., 91-99%, 91-98%, 92-99%, etc.

The terms “stringent conditions” or “stringent hybridization conditions” include conditions under which a probe will hybridize to its target sequence to a detectably greater degree than other sequences (e.g., at least 2-fold over background). Stringent conditions are somewhat sequence-dependent and can vary in different circumstances. By controlling the stringency of the hybridization and/or washing conditions, target sequences can be identified with up to 100% complementarity to the probe (homologous probing). Alternatively, stringency conditions can be adjusted to allow some mismatching in sequences so that lower degrees of sequence similarity are detected (heterologous probing). The probe can be approximately 20-500 nucleotides in length but can vary greatly in length from about 18 nucleotides to equal to the entire length of the target sequence. In some embodiments, the probe is about 10-50 nucleotides in length, or about 18-25 nucleotides in length, or about 18-50 nucleotides in length, or about 18-100 nucleotides in length.

Typically, stringent conditions will be those where the salt concentration is less than about 1.5 M Na⁺ ion (or other salts), typically about 0.01 to 1.0 M Na⁺ ion concentration (or other salts), at pH 7.0 to 8.3 and the temperature is at least about 30° C. for shorter probes (e.g., 10 to 50 nucleotides) and at least about 60° C. for longer probes (e.g., greater than 50 nucleotides). Stringent conditions may also be achieved with the addition of destabilizing agents such as formamide or Denhardt's solution. Exemplary low stringency conditions include hybridization with a buffer solution of 30 to 35% formamide, 1M NaCl, 1% SDS (sodium dodecyl sulfate) at 37° C., and a wash in 1×SSC to 2×SSC (where 20×SSC is 3.0 M NaCl, 0.3 M trisodium citrate) at 50 to 55° C. Exemplary moderate stringency conditions include hybridization in 40 to 45% formamide, 1M NaCl, 1% SDS at 37° C., and a wash in 0.5×SSC to 1×SSC at 55 to 60° C. Exemplary high stringency conditions include hybridization in 50% formamide, 1M NaCl, 1% SDS at 37° C., and a wash in 0.1×SSC at 60 to 65° C. Specificity is typically a function of post-hybridization washes, where the factors controlling hybridization include the ionic strength and temperature of the final wash solution. Thus, high stringency conditions can include a wash that includes 0.1×SSC at 60 to 65° C.

For DNA-DNA hybrids, the Tₘ can be approximated from the equation of Meinkoth and Wahl (Anal. Biochem. 138:267-84 (1984)):

$${T}_{m} = 81.5 ° C . + 16.6 ( log M ) + 0.41 ( \% GC ) - 0.61 ( \% formamide ) - 500 / L$$

where M is the molarity of monovalent cations; % GC is the percentage of guanosine and cytosine nucleotides in the DNA, % formamide is the percentage of formamide in the hybridization solution, and L is the length of the hybrid in base pairs. The Tₘ is the temperature (under defined ionic strength and pH) at which 50% of a complementary target sequence hybridizes to a perfectly matched probe. The Tₘ is reduced by about 1° C. for each 1% of mismatching. Thus, the Tₘ, hybridization and/or wash conditions can be adjusted to hybridize to sequences of the desired sequence identity. For example, if sequences with greater than or equal to 90% sequence identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can include hybridization and/or a wash at 1, 2, 3 or 4° C. lower than the thermal melting point (Tₘ). Moderately stringent conditions can include hybridization and/or a wash at 6, 7, 8, 9 or 10° C. lower than the thermal melting point (Tₘ). Low stringency conditions can include hybridization and/or a wash at 11, 12, 13, 14, 15 or 20° C. lower than the thermal melting point (Tₘ). Using the equation, hybridization and wash compositions, and a desired Tₘ, those of ordinary skill can identify and isolate nucleic acids with sequences related to any of SEQ ID NO: 2, 3, 6, 8, 10, 12, 14, 16, 18, or 22.

Those of skill in the art also understand how to vary the hybridization and/or wash solutions to isolate desirable nucleic acids. For example, if the desired degree of mismatching results in a Tₘ of less than 45° C. (aqueous solution) or 32° C. (formamide solution), it may be preferred to increase the SSC concentration so that a higher temperature can be used.

An extensive guide to the hybridization of nucleic acids is found in Tijssen, LABORATORY TECHNIQUES IN BIOCHEMISTRY AND MOLECULAR BIOLOGY—HYBRIDIZATION WITH NUCLEIC ACID PROBES, part 1, chapter 2, “Overview of principles of hybridization and the strategy of nucleic acid probe assays,” Elsevier, N.Y. (1993); and in CURRENT PROTOCOLS IN MOLECULAR BIOLOGY, chapter 2, Ausubel, et al., eds, Greene Publishing and Wiley-Interscience, New York (1995).

Unless otherwise stated, in the present application high stringency is defined as hybridization in 4×SSC, 5×Denhardt's (5 g Ficoll, 5 g polyvinylpyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C. However, because specificity is typically a function of post-hybridization washes, where the factors controlling hybridization include the ionic strength and temperature of the final wash solution, the high stringency conditions can more simply be expressed as including a wash in 0.1×SSC at 60 to 65° C.

The following terms are used to describe the sequence relationships between two or more nucleic acids or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. The reference sequence can be a nucleic acid sequence (e.g., any of SEQ ID SEQ ID NO:2, 3, 4, or 10) or an amino acid sequence (e.g., any of SEQ ID NO:1, 5, 6, 7, 8, 9, 11, 12, 13, 14, or 15). A reference sequence may be a subset or the entirety of a specified sequence. For example, a reference sequence may be a segment of a full-length cDNA or of a genomic DNA sequence, or the complete cDNA or complete genomic DNA sequence, or a domain of a polypeptide sequence.

As used herein, “comparison window” refers to a contiguous and specified segment of a nucleic acid or an amino acid sequence, wherein the nucleic acid/amino acid sequence can be compared to a reference sequence and wherein the portion of the nucleic acid/amino acid sequence in the comparison window may comprise additions or deletions (i.e., gaps) compared to the reference sequence (which does not comprise additions or deletions) for optimal alignment of the two sequences. The comparison window can vary for nucleic acid and polypeptide sequences. Generally, for nucleic acids, the comparison window is at least 20 contiguous nucleotides in length, and optionally can be 30, 40, 50, 100 or more nucleotides. For amino acid sequences, the comparison window is at least about 10 amino acids, and can optionally be 15, 20, 30, 40, 50, 100 or more amino acids. Those of skill in the art understand that to avoid a high similarity to a reference sequence due to inclusion of gaps in the nucleic acid or amino acid sequence, a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may permit optimal alignment of compared sequences; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG™ programs (Accelrys, Inc., San Diego, Calif.)). The CLUSTAL program is well described by Higgins and Sharp (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. An example of a good program to use for optimal global alignment of multiple sequences is PileUp (Feng and Doolittle, (1987) J. Mol. Evol., 25:351-60, which is similar to the method described by Higgins and Sharp, (1989) CABIOS 5:151-53 (and is hereby incorporated by reference). The BLAST family of programs that can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., eds., Greene Publishing and Wiley-Interscience, New York (1995).

GAP uses the algorithm of Needleman and Wunsch, (1970) J. Mol. Biol. 48:443-53, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP makes a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or more.

GAP presents one member of the family of best alignments. There may be many members of this family. GAP displays four figures of merit for alignments: Quality, Ratio, Identity and Similarity. The Quality is the metric maximized to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

For example, sequence identity/similarity values provided herein can refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (C₁-ayerie and States, (1993) Comput. Chem. 17:191-201) low-complexity filters can be employed alone or in combination.

The terms “substantial identity” indicates that a polypeptide or nucleic acid comprises a sequence with between 55-100% sequence identity to a reference sequence, with at least 55% sequence identity, or at least 60%, or at least 70%, or at least 80%, or at least 90% or at least 95% sequence identity, or at least 96%, or at least 97%, or at least 98%, or at least 99%, or any percentage value within the range of 55-100% sequence identity relative to the reference sequence over a specified comparison window. Optimal alignment may be ascertained or conducted using the homology alignment algorithm of Needleman and Wunsch, supra.

One indication that two CslF6-related polypeptide sequences are substantially identical is that both polypeptides have glucan synthase activity with glucose as a substrate.

The polypeptide that is substantially identical to a CslF6, Fruct4, Tip1-1, or Tip3-1 with a SEQ ID NOS: 1, 5, 7, 9, 11, 13, 15, 17, 19, 20, 21, or 23 sequence may not have exactly the same level of activity as the CslF6, Fruct4, Tip1-1, or Tip3-1 with a SEQ ID NOS: 1, 5, 7, 9, 11, 13, 15, 17, 19, 20, 21, or 23. Instead, the substantially identical polypeptide may exhibit greater or lesser levels of activity than the CslF6 Fruct4, Tip1-1, or Tip3-1 with SEQ ID NOS: 1, 5, 7, 9, 11, 13, 15, 17, 19, 20, 21, or 23, as measured by assays available in the art or described herein (e.g., glucan synthase activity and/or protein folding activity). For example, the substantially identical polypeptide can have at least about 40%, or at least about 50%, or at least about 60%, or at least about 70%, or at least about 80%, or at least about 90%, or at least about 95%, or at least about 97%, or at least about 98%, or at least about 100%, or at least about 105%, or at least about 110%, or at least about 120%, or at least about 130%, or at least about 140%, or at least about 150%, or at least about 200% of the activity of the CslF6 and/or IRE1 with the SEQ ID NO: 1, 5, 7, 9, 11, 13, 15, 17, 19, 20, 21, and 23 sequence when measured by similar assay procedures.

Alternatively, substantial identity is present when second polypeptide is immunologically reactive with antibodies raised against the first polypeptide (e.g., a polypeptide with SEQ ID NOS: 1, 5, 7, 9, 11, 13, 15, 17, 19, 20, 21, and 23). Thus, a polypeptide is substantially identical to a first polypeptide, for example, where the two polypeptides differ only by a conservative substitution. In addition, a polypeptide can be substantially identical to a first polypeptide when they differ by a non-conservative change if the epitope that the antibody recognizes is substantially identical. Polypeptides that are “substantially similar” share sequences as noted above except that some residue positions, which are not identical, may differ by conservative amino acid changes.

The CslF6, Fruct4, Tip1-1, and/or Tip3-1 polypeptides can include the first 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99 N-terminal amino acid residues of a the SEQ ID NO: 1, 5, 7, 9, 11, 13, 15, 17, 19, 20, 21, or 23 sequence. Alternatively, the CslF6, Fruct4, Tip1-1, and/or Tip3-1 polypeptides may include the first 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99 C-terminal amino acid residues of the SEQ ID NO: 1, 5, 7, 9, 11, 13, 15, 17, 19, 20, 21, or 23 sequence.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A fusion protein comprising a vacuolar targeting polypeptide linked to a cellulose synthase like (Csl) protein, wherein the vacuolar targeting polypeptide has an activity of localizing the fusion protein to a tonoplast.

2. The fusion protein of claim 1, wherein the vacuolar targeting polypeptide is a tonoplast intrinsic protein (TIP) protein.

3. The fusion protein of claim 2, wherein the TIP protein comprises a sequence with an amino acid sequence of at least 95% sequence identity to SEQ ID NOS: 15, 17, 19, 20, 21, or 23.

4. The fusion protein of claim 1, wherein the vacuolar targeting polypeptide is a fruc4 vacuole targeting domain.

5. The fusion protein of claim 4, wherein the fruc4 protein comprises a sequence with an amino acid sequence of at least 95% sequence identity to SEQ ID NO: 13.

6. The fusion protein of claim 1, wherein the Csl protein comprises CslF6.

7. The fusion protein of claim 6, wherein the CslF6 protein comprises a sequence with an amino acid sequence of at least 95% sequence identity to SEQ ID NOS: 1, 5, 7, 9, or 11.

8. The fusion protein of claim 1, wherein the vacuolar targeting polypeptide is linked to an N-terminus of the Csl protein.

9. An expression system comprising at least one expression vector comprising a first nucleic acid segment encoding a vacuolar targeting polypeptide linked in-frame with at least one second nucleic acid segment encoding a Csl protein to form a fusion protein, wherein the vacuolar targeting polypeptide has an activity of localizing the fusion protein to a tonoplast.

10. The expression system of claim 9, wherein the vacuolar targeting polypeptide is a tonoplast intrinsic protein (TIP) protein.

11. The expression system of claim 10, wherein the TIP protein comprises a sequence with an amino acid sequence of at least 95% sequence identity to SEQ ID NOS: 15, 17, 19, 20, 21, or 23.

12. The expression system of claim 9, wherein the vacuolar targeting polypeptide is a fruc4 vacuole targeting domain.

13. The expression system of claim 9, wherein the Csl protein comprises CslF6.

14. The expression system of claim 13, wherein the CslF6 protein comprises a sequence with an amino acid sequence of at least 95% sequence identity to SEQ ID NOS: 1, 5, 7, 9, or 11.

15. The expression system of claim 9, wherein the vacuolar targeting polypeptide is linked to an N-terminus of the Csl protein.

16. The expression system of claim 9, wherein the expression vector further comprises a heterologous promotor operably linked to the first nucleic acid segment encoding the vacuolar targeting polypeptide linked in-frame with the at least one second nucleic acid segment encoding the Csl protein.

17. A plant cell, plant seed, or plant comprising the expression system of claim 9.

18. The plant cell, plant seed, or plant of claim 17, wherein the plant cell, plant seed, or plant are selected from the group comprising Brachypodium distachyon, wheat, barely, corn, rice, or sorghum.

19. A method comprising growing a plant seed or plant comprising an expression system comprising a heterologous promoter operably linked to a nucleic acid segment encoding a vacuolar targeting polypeptide linked in-frame with a nucleic acid segment encoding a Csl protein to form a fusion protein, to thereby produce a mature plant.

20. The method of claim 19, further comprising harvesting biomass from the mature plant.

21. The method of claim 20, further comprising isolating glucan, oligosaccharides, disaccharides, monosaccharides, or a combination thereof from the biomass.

22. The method of claim 19, wherein the heterologous promoter is an inducible, or tissue specific promoter.

23. The method of claim 19, wherein the heterologous promoter is selected from a CaMV 35S promoter, CaMV 19S promoter, nos promoter, Adh1 promoter, sucrose synthase promoter, α-tubulin promoter, ubiquitin promoter, actin promoter, cab promoter, PEPCase promoter, R gene complex promoter, poplar xylem-specific secondary cell wall specific cellulose synthase 8 promoter, cauliflower mosaic virus promoter, Z10 promoter from a gene encoding a 10 kDa zein protein, Z27 promoter from a gene encoding a 27 kDa zein protein, pea rbcS gene (Coruzzi et al., EMBO J. 3:1671 (1971)) and the actin promoter from rice promoter, or phaseolin promoter.

24. The method of claim 19, wherein the plant seed or plant are selected from the group comprising Brachypodium distachyon, wheat, barely, corn, rice, or sorghum.

25. A plant cell, plant seed, or plant comprising an expression system comprising a heterologous promoter operably linked to the first nucleic acid segment encoding a Csl protein, wherein the plant cell, plant seed, or plant is Sorghum bicolor.

26. The plant cell, plant seed, or plant of claim 25, wherein the Csl protein is CslF6.

27. The plant cell, plant seed, or plant of claim 26, wherein the CslF6 protein comprises a sequence with an amino acid sequence of at least 95% sequence identity to SEQ ID NOS: 1, 5, 7, 9, or 11.

28. A method comprising growing a plant seed or plant comprising an expression system comprising a heterologous promoter operably linked to a nucleic acid segment encoding a Csl protein to thereby produce a mature plant, wherein the plant cell, plant seed, or plant is Sorghum bicolor, to thereby produce a mature plant.

29. The method of claim 28, further comprising harvesting biomass from the mature plant.

30. The method of claim 29, further comprising isolating glucan, oligosaccharides, disaccharides, monosaccharides, or a combination thereof from the biomass.


SEQUENCE LISTING (23 sequences)

SEQ ID NO: 1 - protein, 939 aa, Brachypodium distachyon
MAPAVAGGSSRGAGCKCGFQVCVCSGSAAVASAGSSLEVERAMAVTPVEGQAAPVDGESWVGVELGPDGVETDESGAGVDDRPVFKTEKIKGVLLHPYRVLIFVRLIAFTLFVIWRISHKNPDTMWLWVTSICGEFWFGFSWLLDQLPKLNPINRIPDLAVLRQRFDRADGTSTLPGLDIFVTTADPIKEPILSTANSVLSILAADYPVDRNTCYISDDSGMLMTYEAMAESAKFATLWVPFCRKHGIEPRGPESYFELKSHPYMGRAHDEFVNDRRRVRKEYDDFKAKINSLETDIQQRNDLHNAAVPQNGDGIPRPTWMADGVQWQGTWVEPSANHRKGDHAGIVLVLIDHPSHDRLPGAPASADNALDFSGVDTRLPMLVYMSREKRPGHNHQKKAGAMNALTRASALLSNAPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPALGGLFAKTKYEKPSMEMTMARANQAVVPAMAKGKHGFLPLPKKTYGKSDKFVDTIPRASHPSPYAAEGIRVVDSGAETLAEAVKVTGSAFEQKTGWGSELGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLATLLIIAVLEVKWAGVTVFEWFRNGQFWMTASCSAYLAAVCQVLTKVIFRRDISFKLTSKLPAGDEKKDPYADLYVVRWTPLMITPIIIIFVNIIGSAVAFAKVLDGEWTHWLKVAGGVFFNFWVLFHLYPFAKGLLGKHGKTPVVVLVWWAFTFVITAVLYINIPHIHGGGGKHSVGHGMHHGKKFDGYYLWP

SEQ ID NO: 2 - DNA, 2820 bp, Brachypodium distachyon
  source 1..2820: mol_type = other DNA
atggcgccagcggtggccggcgggagcagccggggtgcagggtgtaagtgcgggttccaggtgtgcgtgtgctctgggtcggcggcggtggcgtcggcgggttcgtcgctggaggtggagagagccatggcggtgacgccggtggaagggcaggcggcgccggtggacggcgagagctgggtcggcgtcgagctcggccccgacggcgtggagacggacgagagcggcgccggcgtcgacgaccgccccgtcttcaagaccgagaagatcaagggcgtcctcctccacccctacagggtgctgatctttgttcgtctgatagcgttcaccctgttcgtgatctggcgtatctcgcacaagaacccggacacgatgtggctgtgggtgacctccatctgcggcgagttctggttcggcttctcctggctgctggaccagcttccaaagctcaacccgatcaaccggatcccggacctcgccgtgctccggcaacgcttcgaccgcgccgacgggacatccacattgccgggcctcgacatcttcgtcaccacggccgaccccatcaaggaacccatcctgtcgacggccaactccgtgctctccatcctggccgccgactacccggtggaccgcaacacctgctacatctccgacgacagcggcatgctcatgacctacgaggccatggcggagtcggccaagttcgccaccctctgggtgccattctgccgcaagcacggcatcgaaccacgcgggccggagagctacttcgagctcaagtcgcacccgtacatggggagagcgcacgacgagttcgtcaatgaccgccgccgggtgcgcaaggagtatgatgacttcaaggccaagattaactctctggagactgatatccagcagaggaatgatctgcataacgctgccgtgccgcagaatggggatgggatccccaggcctacctggatggctgatggagtccagtggcaggggacttgggtcgagccgtccgctaatcaccgcaagggagaccacgccggcatcgtcctggttctgattgaccacccgagccacgaccgccttcccggcgcgccggcgagcgccgacaacgcgctggacttcagcggcgtggacacccgcctcccgatgctcgtctacatgtcccgcgagaagcgcccaggccacaaccaccagaagaaggccggcgccatgaacgcgctcaccagggcttccgcgctgctctccaacgcgcccttcatcctcaacctcgactgcgaccactacatcaacaactcccaggccctccgcgccgggatctgcttcatggtcggccgggacagcgacaccgtcgccttcgtgcagttcccgcagcggttcgagggcgtcgaccccacggacctctacgccaaccacaaccgcatcttcttcgacggcaccctcagggcgctcgacggaatgcaaggcccgatctatgtcggcacgggatgcctcttccggcgcatcaccgtctacggcttcgacccgcccaggatcaacgtcggcgggccatgcttccctgctctcggtggcctgttcgccaagaccaagtatgagaagcccagcatggagatgaccatggcgagagccaaccaggccgtggtgccggccatggccaaggggaagcacggcttcctgccgctccccaagaagacgtacgggaagtccgacaagttcgtggacaccatcccgcgcgcgtcccacccgtcgccgtacgcggcggaggggatccgcgtggtggactccggcgcggagactctggctgaggccgtcaaggtgaccggatcggcattcgagcagaagaccggatggggcagcgagctcggctgggtctacgacactgtcacagaggacgtggtgactggctacaggatgcacatcaagggctggaggtcccgctactgctccatctacccgcacgccttcatcggcaccgccccgatcaacctcacggagcggctcttccaggtgctccgctggtccaccggctccctcgagatcttcttctccaagaacaacccgctcttcggcagcacctacctgcacccgctccagcgcgtcgcctacatcaacatcaccacatacccgttcaccgccatcttcctcatcttctacaccaccgtgccggcgctctccttcgtcaccggccacttcatcgtgcagcgcccgacgaccatgttctacgtctacctggggatcgtgctggcgacgctgctcatcatcgctgttcttgaggtcaagtgggctggagtgacagtgttcgagtggttcaggaacgggcagttctggatgacggctagctgctccgcctaccttgctgctgtgtgccaggtgctcaccaaggtgatcttcaggagggacatctcattcaagctcacttccaagctgcctgctggggacgagaagaaggacccctatgccgatctgtacgtggtgcgttggactccactcatgatcactccaatcatcatcatcttcgtcaacatcatcggctcggcggtggccttcgccaaggtgctggacggcgagtggacgcactggctcaaggtggcgggaggagtcttcttcaacttctgggtgctgttccacctctacccgttcgccaagggtctcctggggaagcatggcaagacccccgtcgtcgtgctcgtctggtgggcattcaccttcgtcatcaccgccgtcctctacatcaacatcccgcacatccatggaggaggaggcaagcacagcgtggggcatgggatgcaccatggcaagaagttcgacggctactacctctggccgtga

SEQ ID NO: 3 - DNA, 2820 bp, Brachypodium distachyon
  source 1..2820: mol_type = other DNA
atggctccagctgttgctggcggctcctctaggggcgctggctgcaagtgcggcttccaggtgtgcgtgtgctccggctctgccgccgtggcctccgccggctcatccctcgaggtcgagagggccatggctgttaccccagttgagggccaggccgctccagtggacggcgagtcctgggtgggcgttgagcttggcccagacggcgtcgagaccgacgagtccggcgctggcgtggacgacaggccagtgttcaagaccgagaagatcaagggcgtgctcctccacccatacagggtgctcatcttcgtgaggctgatcgccttcaccctcttcgtgatctggcgcatctcccacaagaacccggacaccatgtggctctgggtgacctctatttgcggcgagttctggttcggcttctcctggctcctcgaccagctcccaaagctcaacccgatcaaccgcatcccagatctcgccgttctcaggcagaggttcgatagggccgacggcacctccaccctcccaggccttgatattttcgtgaccaccgccgaccccatcaaggagccaattctctcaaccgccaactccgtgctctctatcctcgccgccgattacccggtggataggaacacgtgctacatctccgacgacagcggcatgctcatgacctacgaggctatggccgagtccgccaagttcgctaccctctgggtgccattctgccgcaagcacggcatcgagccaaggggcccagagtcctacttcgagcttaagtcccacccgtacatgggcagggcccatgacgagttcgtgaacgataggcgcagggtgaggaaggagtacgacgacttcaaggccaagatcaactccctcgagacggacatccagcagaggaacgacctccataacgccgccgtgccacagaacggggacggcatcccaaggccaacctggatggccgatggcgtgcagtggcagggcacctgggttgagccatctgccaaccataggaagggcgatcacgccggcattgtgctcgtgctcatcgaccatccatcccacgacaggctcccaggcgccccagcctctgccgacaacgccctcgacttctccggcgtggacaccaggcttccaatgctcgtttacatgtcccgcgagaagaggccaggccacaaccaccagaagaaggctggcgctatgaacgcccttaccagggcttctgctctcctctccaacgccccgttcatcctcaacctcgactgcgaccactacatcaacaacagccaggctctcagggccggcatctgcttcatggtgggcagggattctgacaccgtggccttcgttcagttcccgcagcgcttcgagggggttgacccaaccgatctctacgccaaccacaacaggattttcttcgatggcaccctcagggccctcgatggcatgcagggccctatctacgtgggcaccggctgcctcttcaggcgcatcaccgtgtacggcttcgacccgccaaggattaacgttggcggcccatgcttcccagctctcggcggcctcttcgctaagaccaagtacgagaagcccagcatggagatgaccatggccagggccaaccaggccgttgttccagctatggctaaggggaagcacggcttcctgccactcccgaagaagacctacggcaagagcgacaagttcgtcgacaccattccaagggcctcccacccatctccatacgctgccgagggcattagggttgtggactctggcgccgagaccctcgccgaggccgtgaaggtgaccggctccgccttcgagcagaagaccggctggggctccgagcttggctgggtttacgacaccgtgaccgaggatgtggtcaccggctacaggatgcacattaagggctggcgcagcaggtactgctccatctacccacatgccttcatcggcaccgcccccattaacctcaccgagaggcttttccaggtgctcaggtggtctaccggcagcctcgagatcttcttcagcaagaacaacccgctgttcggctccacctacctgcatccactccagagggtggcctacattaacatcaccacctacccgttcaccgccatcttcctcatcttctacacgaccgtgcccgccctctcattcgtgaccggccatttcattgtgcagaggccgaccaccatgttctacgtgtacctcgggatcgtgctcgccaccctcctcattattgccgtgctcgaggttaagtgggctggcgtgaccgtgttcgagtggttccgcaacggccagttctggatgaccgcctcttgctctgcttacctcgccgctgtttgccaggtcctcaccaaggttatcttccgcagggacatctccttcaagctcacctccaagctcccagccggcgacgagaagaaggacccatacgccgatctgtacgtggtgaggtggaccccgctcatgatcaccccgatcatcatcattttcgtcaacatcatcggctccgcggtcgccttcgccaaggtgctcgatggcgagtggacccattggcttaaggtcgccggcggcgtgttcttcaacttctgggttctcttccacctctaccctttcgcgaagggccttcttggcaagcacggcaagaccccagtggtggttcttgtctggtgggccttcaccttcgtcatcaccgccgtgctgtacatcaacatcccgcacatccatggcggcggcggcaagcactccgtgggccacggcatgcaccatggcaagaagttcgacggctactacctctggccgtga

SEQ ID NO: 4 - skipped

SEQ ID NO: 5 - protein, 944 aa, Triticum aestivum
MAPAVAGGGRVRSNEPAAAATAPASGKPCVCGFQVCACTGSAAVASAASSLDMDIVAMGQIGAVNDESWVGVELGEDGETDESGAAVDDRPVFRTEKIKGVLLHPYRVLIFVRLIAFTLFVIWRISHKNPDAMWLWVTSICGEFWFGFSWLLDQLPKLNPINRVPDLAVLRQRFDRPDGTSTLPGLDIFVTTADPIKEPILSTANSVLSILAADYPVDRNTCYVSDDSGMLLTYEALAESSKFATLWVPFCRKHGIEPRGPESYFELKSHPYMGRAQDEFVNDRRRVRKEYDEFKARINSLEHDIKQRNDGYNAANAHREGEPRPTWMADGTQWEGTWVDASENHRRGDHAGIVLVLLNHPSHRRQTGPPASADNPLDFSGVDVRLPMLVYMSREKRPGHDHQKKAGAMNALTRASALLSNSPFILNLDCNHYINNSQALRAGICFMVGRDSDTVAFVQFPQRFEGVDPTDLYANHNRIFFDGTLRALDGMQGPIYVGTGCLFRRITVYGFDPPRINVGGPCFPRLAGLFAKTKYEKPGLEMTMAKAKAAPVPAKGKHGFLPLPKKTYGKSDAFVDSIPRASHPSPYAAAAEGIVADEATIVEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSKLPSGDEKKDPYADLYVVRWTPLMITPIIIIFVNIIGSAVAFAKVLDGEWTHWLKVAGGVFFNFWVLFHLYPFAKGILGKHGKTPVVVLVWWAFTFVITAVFYINIPHMHSSGGKHTTVHGHHGKKFVDAGYYNWP

SEQ ID NO: 6 - DNA, 2925 bp, Triticum aestivum
  source 1..2925: mol_type = other DNA
atggcgccagcggtggccggaggaggccgcgtgcggagcaacgagccggctgctgccgctaccgcgcccgccagcgggaagccctgcgtctgcggcttccaggtgtgcgcctgcacgggatcggccgcggtggcgtctgccgcctcctcgctcgacatggacatcgtggccatggggcagataggcgccgtcaacgacgagagctgggtgggcgtggagctcggcgaggacggcgagaccgacgaaagcggtgccgccgttgacgaccgccccgtcttccgcaccgagaaaatcaagggtgtcctcctccacccctaccgggtgctgatcttcgtccgtctcatcgccttcaccctgttcgtgatctggcgtatttcccacaagaaccctgacgccatgtggctgtgggtgacatccatctgcggcgagttctggttcggtttctcctggctgctggatcagctccccaagctgaaccccatcaaccgcgtgccggacctggccgtgctgcggcagcgcttcgaccgccccgacggcacctccaccctcccggggctggacatcttcgtcaccacggccgaccccatcaaggagcccatcctctccactgccaactcggtgctctccatcctcgccgccgactaccccgtcgaccgcaacacatgctacgtttctgacgacagtggcatgctgctcacctacgaggccctggctgagtcctccaagttcgccaccctctgggtgcccttctgccgcaagcacgggatcgagcccaggggcccggagagctactttgagctcaagtcccacccctacatggggagagcccaggacgagttcgtgaacgaccgtcgccgcgtccgcaaggagtacgacgagttcaaggccaggatcaacagcctggagcatgacatcaagcagcgcaacgacgggtacaacgccgccaacgcccaccgggaaggcgagccccgacccacctggatggccgacggcacccagtgggagggcacctgggtcgacgcctccgagaaccaccgcaggggcgaccacgccggcatcgtcctggtgctgctgaaccacccgagccaccgccggcagacgggcccgccggcgagtgctgacaacccactggacttcagcggcgtggatgtgcgtctccccatgctggtgtacgtgtcccgtgagaagcgccccggacacgaccaccagaagaaggccggcgccatgaacgcgctcacccgcgcctccgcgctgctctccaactcccccttcatcctcaacctcgactgcgaccactacatcaacaactcccaggccctccgcgccggcatctgcttcatggtgggacgcgacagcgacaccgtcgccttcgtccagttcccgcagcgcttcgagggcgtcgaccccaccgacctctacgccaaccacaaccgcatcttcttcgacggcaccctccgtgccctcgacggcatgcagggccccatctacgtcggcaccggctgtctcttccgccgcatcaccgtctacggcttcgacccgcccaggatcaacgtcggcgggccctgcttccccaggctcgccgggctcttcgccaagaccaagtacgagaagcctgggctcgagatgaccatggccaaggccaaggctgcgccggtgcccgccaagggcaagcacggcttcctgcctctgcccaagaagacgtacggcaagtcggacgccttcgtggacagcatcccgcgcgcgtcgcacccgtcgccttacgccgcggcggctgagggcatcgtggccgacgaggcgaccatcgtggaggcggtgaacgtgacggcggcggcgttcgagaagaagaccgggtggggcaaagagatcggctgggtgtacgacaccgtgacggaggacgtggtgaccgggtaccggatgcatatcaaggggtggcggtcacgctactgctccatctacccacacgccttcatcggcaccgcacccatcaacctcacggagaggctcttccaggtgcttcgctggtccacgggctccctcgagatcttcttctccaagaacaacccgctcttcggcagcacctacctccacccgctgcagcgcgtcgcctacatcaacatcaccacctacccgttcaccgccatcttcctcatcttctacaccaccgtgccggcgctctccttcgtcactggccacttcatcgtgcagcgccccaccaccatgttctacgtctacctgggcatcgtgctctccacgctgctcgtcatcgccgtgctggaggtcaagtgggccggggtcaccgtcttcgagtggttcaggaacggccagttctggatgacggcaagttgctccgcctacctcgccgccgtgtgccaggtgctcaccaaggtgatattccggcgtgacatctccttcaagctcacatccaagctaccgtcgggagacgagaagaaggacccctacgccgacctctacgtggtgcgctggacgccgctcatgatcacacccatcatcatcattttcgtcaacatcatcgggtcggcggtggccttcgccaaggtgctcgacggcgagtggacgcactggctcaaggtcgccggcggcgtcttcttcaacttctgggtgctcttccacctctacccgttcgccaagggcatcctggggaagcacggcaagacgccagtcgtggtgctcgtctggtgggcattcaccttcgtcatcaccgccgtgctctacatcaacatcccccacatgcatagctcgggaggcaagcacacaacggtgcatggtcaccatggcaagaagttcgtcgacgcagggtactacaactggccgtgaggtccctgttgacgactttgccgccgcgcatccggacaagacaacgacgacctgcgacaagaaacaacaagtcatccactgaacagtgca

SEQ ID NO: 7 - protein, 947 aa, Hordeum vulgare
MAPAVAGGGRVRSNEPVAAAAAAPAASGKPCVCGFQVCACTGSAAVASAASSLDMDIVAMGQIGAVNDESWVGVELGEDGETDESGAAVDDRPVFRTEKIKGVLLHPYRVLIFVRLIAFTLFVIWRISHKNPDAMWLWVTSICGEFWFGFSWLLDQLPKLNPINRVPDLAVLRQRFDRPDGTSTLPGLDIFVTTADPIKEPILSTANSVLSILAADYPVDRNTCYVSDDSGMLLTYEALAESSKFATLWVPFCRKHGIEPRGPESYFELKSHPYMGRAQDEFVNDRRRVRKEYDEFKARINSLEHDIKQRNDGYNAAIAHSQGVPRPTWMADGTQWEGTWVDASENHRRGDHAGIVLVLLNHPSHRRQTGPPASADNPLDLSGVDVRLPMLVYVSREKRPGHDHQKKAGAMNALTRASALLSNSPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPRLAGLFAKTKYEKPGLEMTTAKAKAAPVPAKGKHGFLPLPKKTYGKSDAFVDTIPRASHPSPYAAAAEGIVADEATIVEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSKLPSGDEKKDPYADLYVVRWTPLMITPIIIIFVNIIGSAVAFAKVLDGEWTHWLKVAGGVFFNFWVLFHLYPFAKGILGKHGKTPVVVLVWWAFTFVITAVLYINIPHMHTSGGKHTTVHGHHGKKLVDTGLYGWLH

SEQ ID NO: 8 - DNA, 3307 bp, Hordeum vulgare
  source 1..3307: mol_type = other DNA
cgtaaaggagagtgagtgcgtgcattgaggacgacggccatggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccgggtgctgattttcgttcgtctgatcgccttcacgctgttcgtgatctggcgtatctcccacaagaacccagacgcgatgtggctgtgggtgacatccatctgcggcgagttctggttcggtttctcgtggctgctagatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattgatgactttgcccggacaagacgacctgagacaagaaacaactcatccactcaacagtcagtgcatgcatccatctcatcgagaagcagagcccgccaaagtttgaattttttaattttttttcttcacttttttgcccgtttctttttagttttgtccagaaaaaagatggtgttgatttgatttagtttataattacctgtggtaattaattatgtacttaattatacattccgcgaacaacaagggagacagacgacttacggggtactggctcgggtggtaagagcttgcactgtactgtacatgctcgacgatgtatagagatgcacagaggagaggatgggagtgctgggaccgtggggtggacggcggtattcttttagtattatatatggaaacaataaatttaatttcattaaaaaa

SEQ ID NO: 9 - protein, 917 aa, Zea mays
MAAVASAASSADMDRVAVAATEGQIGAVNDESWIAVDLSDDGLSADGADPGVALEDRPVFRTEKIKGVLLHPYRVLIFVRLIAFTLFVIWRISHRNPDALWLWVTSIAGEFWFGFSWLLDQLPKLNPINRVPDLAALRQRFDRAGGGAGGGTSLLPGLDVFVTTADPFKEPILSTANSVLSILAADYPVERNTCYLSDDSGMLLTYEAMAEAAKFATVWVPFCRKHGIEPRGPESYFDLKSHPYMGRSQEDFVNDRRRVRKDYDEFKARINGLDHDIKQRSDAYNAARGLKDGEPRATWMADGTQWEGTWVEPSENHRKGDHAGIVLVLLNHPSHSRQLGPPASADNPLDLSMVDVRLPMLVYVSREKRPGHNHQKKAGAMNALTRCSAVLSNSPFILNLDCDHYINNSQALRAGICFMLGRDSDTVAFVQFPQRFEGVDPTDLYANHNRIFFDGTLRALDGMQGPIYVGTGCLFRRITLYGFDPPRINVGGPCFPALGGMFAKAKYEKPGLELTTKAAVAKGKHGFLPMPKKSYGKSDAFADTIPMASHPSPFAAAAAAVVVADEATIAEAVAVCAAAYEKKTGWGSDIGWVYGTVTEDVVTGYRMHIKGWRSRYCSIYPHAFIGTAPINLTERLFQVLRWSTGSLEIFFSRNNPLFGSTFLHPLQRVAYINITTYPFTAIFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSKQPAGDEKKDPYADLYVVRWTWLMVTPIIIILVNIIGSAVAFAKVLDGEWTHWLKVAGGVFFNFWVLFHLYPFAKGILGRHGKTPVVVLVWWAFTFVITAVLYINIPHIHGPGGKHGGAIGRHGGDAHHHGKKFDGYYLWP

SEQ ID NO: 10 - DNA, 2795 bp, Zea mays
  source 1..2795: mol_type = other DNA
cttccgccaggcaggcagagagggagggcgcgcccggcattatggcggcggtggcgtccgccgcgtcctccgccgacatggaccgcgtggccgtggccgccaccgagggccagatcggcgccgtcaacgacgagagctggatcgccgtcgacctcagcgacgacggcctgtccgccgacggcgccgacccgggcgtcgcgctcgaggaccgccccgtcttccgcaccgagaagatcaagggcgtcctcctccacccctacagggtgctgatcttcgtccggctgatcgcgttcacgctgttcgtgatctggcgcatctcgcaccgcaacccggacgcgctgtggctgtgggtgacgtcgatcgcgggcgagttctggttcggcttctcgtggctgctggaccagctgccgaagctgaacccgatcaaccgcgtgccggacctggcggcgctgcggcagcggttcgaccgcgcgggcgggggcgccggcggagggacgtcgctgctgccggggctggacgtgttcgtgacgacggcggacccgttcaaggagccgatactgagcacggccaactccgtgctgtccatcctggccgccgactaccccgtggagcgcaacacgtgctacctctccgacgactccgggatgctgctcacctacgaggccatggcggaggccgccaagttcgccaccgtctgggtgcccttctgccgcaagcacggcatcgagccgcgcggccccgagagctacttcgacctcaagtcccacccttacatgggccgctcccaggaggacttcgtcaacgaccgccgccgcgtgcgcaaggactacgacgagttcaaggcgcgcatcaacggcctcgaccacgacatcaagcagaggtccgacgcgtacaacgccgccagggggctcaaggacggcgagcccagggcaacctggatggccgacggcacccagtgggagggcacctgggtcgagccctccgagaaccaccgcaagggagaccacgccggcatcgtactggtgcttctgaaccacccgagccacagccgtcagctcggcccgccggcgagcgctgacaacccgctggacttgagcatggtggacgtgcggctcccgatgctggtgtacgtgtcccgcgagaagcggcccggtcacaaccaccagaagaaggccggcgccatgaacgcgctgacccggtgctccgcggtgctgtccaactcgcccttcatcctcaacctggactgcgaccactacatcaacaactcgcaggcgctgcgcgcgggcatctgcttcatgctcggccgcgacagcgacacggtggccttcgtgcagttcccgcagcgcttcgagggcgtggaccccacggacctgtacgccaaccacaaccgcatcttcttcgacggcacgctcagggcgctggacggcatgcagggccccatctacgtgggcacgggctgcctgttccgccgcatcacgctctacggcttcgacccgccgcggatcaacgtgggcgggccgtgcttcccggcgctgggcggcatgttcgccaaggccaagtacgagaagcccggactggagctcaccaccaaggccgccgtggccaagggcaagcacggcttcctgcccatgcctaagaagtcgtacggcaagtcggacgcgttcgcggacaccatcccgatggcgtcgcacccgtcgccgttcgcggcggccgccgccgccgtcgtcgtggcggacgaggcgaccatcgccgaggcagtggcggtgtgcgcggcggcgtacgagaagaagaccgggtggggcagcgacatcggatgggtgtacggcacggtgacggaggacgtggtgacggggtaccggatgcacatcaaggggtggcggtcccgctactgctccatctacccgcacgccttcatcggcaccgcccccatcaacctgacggagcggctgttccaggtgctccgctggtccacgggttccctggagatcttcttctcccggaacaacccgctgttcgggagcacgttcctgcacccgctgcagcgcgtggcgtacatcaacatcacgacgtacccgttcacggccatcttcctcatcttctacacgacggtgccggcgctgtcgttcgtgacggggcacttcatcgtgcagcggcccaccaccatgttctacgtgtacctggccatcgtgctgggcacgctgctgatcctggccgtcctggaggtgaagtgggcgggcgtgacggtgttcgagtggttcaggaacgggcagttctggatgacggccagctgctccgcgtacctggccgcggtgtgccaggtgctggtgaaggtggtgttcaggcgggacatctcgttcaagctgacgtcgaagcagcccgcgggggacgagaagaaggacccctacgcagacctgtacgtggtgcgctggacctggctcatggtgacgcccatcatcatcatcctcgtcaacatcatcggctccgccgtggccttcgccaaggtgctggacggcgagtggacgcactggctcaaggtggccggcggcgtcttcttcaacttctgggtgctcttccacctctaccctttcgccaagggcatcctggggaggcacggcaagacccccgtggtggtgctcgtctggtgggccttcaccttcgtcatcaccgccgtgctgtacatcaacatcccacacatccacgggcccggcggcaagcacggcggcgcgatcggaaggcacggcggcgacgcacaccaccatggcaagaagttcgacggctactacctctggccgtga

SEQ ID NO: 11 - protein, 961 aa, Sorghum bicolor
MAPGGGDGRRNGEGQQQANGNNNNNNSNAKAKHGCVCGFPVCACAGAAAVASAASSADMDRVAAAQTEGQIGAVNDESWIAVDLSDDLSGDGGGADPGVAIEDRPVFRTEKIKGILLHPYRVLIFVRLIAFTLFVIWRISHRNPDAMWLWVTSIAGEFWFGFSWLLDQLPKLNPINRVPDLAVLRQRFDRADGTSRLPGLDIFVTTADPFKEPILSTANSILSILAADYPVERNTCYLSDDSGMLLTYEAMAEAAKFATVWVPFCRKHGIEPRGPESYFELKSHPYMGRSQEDFVNDRRRVRKEYDEFKARINGLEHDIKQRSDAFNAARGLKDGEPRATWMADGNQWEGTWVEPSENHRKGDHAGIVYVLLNHPSHSRQLGPPASADNPLDFSMVDVRLPMLVYVSREKRPGFNHEKKAGAMNALTRCSAVISNSPFILNLDCDHYINNSQALRAGICFMLGRDSDTVAFVQFPQRFEGVDPTDLYANHNRIFFDGTLRALDGMQGPIYVGTGCMFRRITLYGFDPPRINVGGPCFPSLGGMFAKTKYEKPGLELTTKAAVAKGKHGFLPLPKKSYGKSDAFVDTIPRASHPSPFLSADEAAAIVADEAMITEAVEVCTAAYEKKTGWGSDIGWVYGTVTEDVVTGYRMHIKGWRSRYCSIYPHAFIGTAPINLTERLYQVLRWSTGSLEIFFSRNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSKQPAGDEKKDPYADLYVVRWTWLMVTPIIIILVNIIGSAVAFAKVLDGEWTHWLKVAGGVFFNFWVLFHLYPFAKGLLGRHGKTPVVVLVWWAFTFVITAVLYINIPHIHGPGGKHGGAIGKHGAAHHGKKFDLDNLSYNWP

SEQ ID NO: 12 - DNA, 2906 bp, Sorghum bicolor
  source 1..2906: mol_type = other DNA
gagggcgcagccggcattatggcgccgggcggcggagacggccggcgcaacggcgagggacagcagcaggcgaacggcaacaacaacaacaacaacagcaacgctaaggctaagcacgggtgcgtgtgcgggttccccgtgtgcgcgtgcgccggcgcggccgcggtggcgtccgcggcctcctccgccgacatggaccgcgtggccgccgcgcagaccgagggccagatcggcgccgtcaacgacgagagctggatcgccgtcgacctgagcgacgacctctccggcgacggcggcggcgccgaccccggcgtcgcgatcgaggaccgccccgtcttccgcaccgagaagatcaagggcatcctcctccacccctacagggtgctcatcttcgtgcgcctgatcgcgttcacgctgttcgtcatctggcgtatctcgcaccgcaacccggacgcgatgtggctgtgggtgacgtcgatcgcgggcgagttctggttcggcttctcctggctgctggaccagctccccaagctgaacccgatcaaccgcgtcccggacctcgcggtgctccggcagcggttcgaccgcgccgacggcacgtcccgcctcccgggcctggacatcttcgtcaccacggcggacccgttcaaggagcccatcctgagcacggccaactccatcctctccatccttgccgccgactaccccgtggagcgcaacacgtgctacctctccgacgactccgggatgctgctcacctacgaggccatggcggaggccgccaagttcgccaccgtctgggtgcccttctgccggaagcacggcatcgagcctcgtggccccgagagctacttcgagctcaagtcgcacccctacatggggaggtcgcaggaggacttcgtcaacgaccgccgccgtgtccgcaaggagtacgacgagttcaaggcccggatcaatggcctcgagcatgatatcaagcagaggtccgacgcgtttaacgccgctagggggcttaaggacggcgagcccagagctacgtggatggccgacgggaaccagtgggagggcacatgggttgagccatcggagaaccaccgcaagggtgaccacgccggcatcgtctatgtgcttctgaaccacccgagccacagccgtcagctcggcccgccggcgagcgcggacaacccgctggacttcagcatggtggacgttcgcctccccatgctggtgtacgtctcccgtgagaagcggcccgggttcaaccacgagaagaaggccggcgccatgaacgcgctgacccgctgctccgccgtgatctccaactcgcccttcatcctcaacctggactgcgaccactacatcaacaactcgcaggcgcttcgcgccggcatctgcttcatgctcggccgggacagcgacacggtggcgttcgtgcagttcccgcagcggttcgagggcgtggaccccacggacctgtacgccaaccacaaccgcatcttcttcgacggcacgctccgggcgctggacggcatgcagggccccatctacgtcggcaccggctgcatgttccgccgcatcacgctctacggcttcgacccgccgaggatcaacgtcggcgggccgtgcttcccgtcgctcggcggcatgttcgccaagaccaagtacgagaagcccgggctggagctcaccaccaaggccgccgtcgccaagggcaagcacggcttcctcccgttgcccaagaagtcgtacggcaagtcggacgcgttcgtcgacaccatcccgagggcgtctcacccgtcgccgttcctgagcgccgacgaggccgccgccatcgtcgccgacgaggccatgatcaccgaggccgtggaggtgtgcacggcggcgtacgagaagaagaccggctggggcagcgacatcggctgggtgtacggcaccgtcaccgaggacgtggtgacggggtaccggatgcacatcaaggggtggcggtctcgctactgctccatctacccgcacgccttcatcggcaccgccccgatcaacctgacggagcggctgtaccaggtgctccgctggtccacggggtcgctggagatcttcttctcccggaacaacccgctgttcggcagcacgttcctgcacccgctgcagcgcgtggcgtacatcaacatcaccacctacccgttcacggcgctgttcctcatcttctacaccaccgtgccggcgctgtcgttcgtgacggggcacttcatcgtgcagcggccgaccaccatgttctacgtgtacctggccatcgtgctggggacgctgctcatcctggccgtcctggaggtgaaatgggcgggcgtcaccgtcttcgagtggttcaggaacgggcagttctggatgacggccagctgctccgcgtacctggccgccgtgtgccaggtgctggtgaaagtggtgttccggcgagacatctccttcaagctcacatccaagcagcccgccggcgacgagaagaaggacccctacgccgacctgtacgtggtgcgctggacctggctcatggtgacccccatcatcatcatcctcgtcaacatcatcggatccgccgtggcgttcgccaaggtgctggacggcgagtggacgcactggctcaaggtggccggcggcgtcttcttcaacttctgggtgctgttccacctctacccgttcgccaagggcctcctcgggaggcacggcaagacccccgtggtggtgctcgtctggtgggcattcaccttcgtcatcaccgccgtgctctacatcaacatcccccacatccatggccccggcggcaagcacggcggcgcgatcggaaagcacggcgccgcccaccacggcaagaagttcgacctcgacaacctctcctacaactggccgtgaag

SEQ ID NO: 13 - protein, 44 aa, Arabidopsis thaliana
MASSDALLPISAREEEPLCPYTRLPMADPNQETHGPRRRRPFKG

SEQ ID NO: 14 - DNA, 142 bp, Arabidopsis thaliana
  source 1..142: mol_type = other DNA
cgatgctctcttgccaatctccgccagagaagaagaaccattatgtccttacacgagattaccaatggccgacccgaatcaagaaacccatggcccccggagaagaagaccctttaaaggtctcctcgccgtctcatttggt

SEQ ID NO: 15 - protein, 251 aa, Arabidopsis thaliana
MPIRNIAIGRPDEATRPDALKAALAEFISTLIFVVAGSGSGMAFNKLTENGATTPSGLVAAAVAHAFGLFVAVSVGANISGGHVNPAVTFGAFIGGNITLLRGILYWIAQLLGSVVACLILKFATGGLAVPAFGLSAGVGVLNAFVFEIVMTFGLVYTVYATAIDPKNGSLGTIAPIAIGFIVGANILAGGAFSGASMNPAVAFGPAVVSWTWTNHWVYWAGPLVGGGIAGLIYEVFFINTTHEQLPTTDY

SEQ ID NO: 16 - DNA, 993 bp, Arabidopsis thaliana
  source 1..993: mol_type = other DNA
gctcttctagcatcttgaagcttaaatctcagccgtccgatcatgccgatcagaaacatcgccattggccgtccagatgaagccacccgtcccgatgccttaaaggcggcgttggctgagttcatttcaactttgatctttgtcgtcgccggttcaggctctggcatggctttcaacaagctcactgaaaacggagccaccactccttctggtctcgtagctgctgcagtggctcatgcctttggactcttcgtcgctgtctcagttggtgccaacatctctggtggacacgttaaccctgccgtcactttcggtgctttcattggtggtaacatcactctcctccgtggtatcctctactggattgctcagcttctcggctccgtcgtcgcttgcctcatccttaaattcgccaccggaggcttggctgtgcccccttttggtctctctgctggagtaggagtgttgaacgctttcgttttcgagatcgtgatgacattcgggcttgtttacaccgtctacgctacagccattgaccccaaaaacgggagtcttggaacaattgctcccatcgcaatcggtttcattgttggagccaacatcttagctggaggagctttctctggagcctccatgaatcccgccgtggctttcggaccagcggtggtgagctggacatggaccaaccactgggtctactgggccggacctctcgtcggcggtggaatcgctggactcatctacgaagttttcttcatcaacaccacacacacgagcagctccaaccacagactactgaattaatctctctttttctctcttgtgtaattttattcgactttgaatttgaattttaatgtctttttaatttccgtttgtgtaatttcgaatcatcaaggggtttatgatcgttcgatcttttagatgaatccttgtccgttggtttcaagagtggccattgcaatttcgactatataatgtgaattatgtatc

SEQ ID NO: 17 - protein, 268 aa, Arabidopsis thaliana
MATSARRAYGFGRADEATHPDSIRATLAEFLSTFVFVFAAEGSILSLDKLYWEHAAHAGTNTPGGLILVALAHAFALFAAVSAAINVSGGHVNPAVTFGALVGGRVTAIRAIYYWIAQLLGAILACLLLRLTTNGMRPVGFRLASGVGAVNGLVLEIILTFGLVYVVYSTLIDPKRGSLGIIAPLAIGLIVGANILVGGPFSGASMNPARAFGPALVGWRWHDHWIYWVGPFIGSALAALIYEYMVIPTEPPTHHAHGVHQPLAPEDY

SEQ ID NO: 18 - DNA, 998 bp, Arabidopsis thaliana
  source 1..998: mol_type = other DNA
acactttgttgttaatttcaagtgtttgatcataatggcaacatcagctcgtagagcatacggtttcggtagagccgatgaggctacacaccctgactccattagagcaactttagctgagtttctctccacttttgtcttcgtctttgcagctgaaggctctatcctctctctcgataagttgtattgggaacatgcggctcatgcggggacaaacacaccaggagggctgattttagtagcgttggctcatgcgtttgctctgtttgctgctgtttcagcagccattaatgtctccggcggacacgttaacccggcagtcacttttggtgctcttgttggaggcagagttacagcgatccgcgccatctactactggatcgctcagcttcttggagccatcctcgcttgtctcttgttaaggctcacaacaaacggcatgagaccagttggtttccgtctagcatcaggtgttggagcggttaatggacttgtattagagatcattttaacatttggcttagtctacgtagtgtattccactttgattgatccaaaacgtggaagcctcgggatcatagcaccgcttgcaatcggactcatagttggggcaaacatcttagtaggtggaccattttctggtgcttcgatgaatccagctagagcttttggtccagcgttggtgggatggagatggcatgaccactggatctattgggtcggaccattcatcggtagtgctttagccgcccttatatatgagtacatggtcatacccaccgaaccacctacccaccacgcacatggtgtacaccagcccttggcccctgaagattactagatggaactttcctcatgtcaccactgcttttgttcgttgttcaagctctctttgtctgtatgatgagatcacttgcatagatactttttttcttatgctactcttatgttgtaataataaaaaataaagaacgatttcgctaaaaaaaaaaaaaaaa

SEQ ID NO: 19 - protein, 252 aa, Brachypodium distachyon
MPVSRIAIGSPGELSHPDTFKAAVAEFISMLIFVFAGSGSGMAFGKLTDGGATTPSGLIAAALAHAFALFVAVSVGANISGGHVNPAVTFGAFIGGNISLLKAIVYWVAQLLGSTAACLLLQISTGGASVGAFSLSAGVGVWNAVVFEIVMTFGLVYTVYATAVDPKRGDLGVIAPIAIGFIVGANILAGGAFDGASMNPAVSFGPAVVSGVWENHWVYWLGPFAGAAIAALVYDIVFIGQRPHDQLPSAEY

SEQ ID NO: 20 - protein, 250 aa, Brachypodium distachyon
MPISRVAVGSHHEVYQAGALKAAVAEFISTLIFVFAGQGSGMAFNKLNAGGAATPAGLISAAIAHAFALFVAVSVGANISGGHVNPAVTFGAFLGGNITLFRGLLYWVAQLLGSTAACFLLRFSTGGLPTGTFGLTGVSVWEALVLEIVMTFGLVYTVYATAVDPKKGSIGTIAPLAIGFIVGANILVGGAFDGASMNPAVSFGPALVSWEWGYQWVYWVGPLIGGGLAGVIYEVLFISHTHEQLPTTDY

SEQ ID NO: 21 - protein, 131 aa, Triticum aestivum
EFISTLIFVFAGQGSGMAFSKLSPDGAATPAGLISAAIAHAFALFVAVSVGANISGGHVNPAVTFGAFVGGNITLFRGLLYWIAQLLGSTAACFLLRFSTGGLPTGTFGLSGIGAWEAVVLEIVMTFGLVY

SEQ ID NO: 22 - DNA, 1092 bp, Triticum aestivum
  source 1..1092: mol_type = other DNA
acacgagccgatccatccatccagaaaaaaaaaaaagagtgcccgtcgaaaatgccggtgcgcaggatcgccatcggcgccccgggggagctgtcccacccggacaccttccgcgccggcgtcgccgagttcatctccatgctcatcttcgtcttcgccggctcaggctccggcatggccttcggcaagctgactgacggcggcgcgacgacgccggcggggcttatcgcggcggcgctggcgcacgcgttcgcgctcttcgtggccgtctccgtgggcgccaacatctccggcgggcacgtgaacccggccgtcacattcggcgccttcgtgggcggcaacatcagcctgctcaaggccgtggtgtactgggtggcgcagctgctcggctccgtcgtggcctgcctcctcctcaagatcgccaccggcggcgaggccgtgggcgccttctcgctctccgccggcgtcggcgtctggaacgccgtggtgttcgagatcgtcatgaccttcgggctcgtctacacggtgtacgccaccgcggtcgaccccaagcgcggcgacctcggggtgatcgcgcccatcgccatcggcttcatcgtcggcgccaacatcctcgccggcggcgccttcgacggcgcctccatgaaccccgccgtctccttcggccccgctgtcgtcagcggcgtctgggagaaccactgggtctactggctcggccccttcgtcggcgccgccatcgccgccctcgtctacgacatctgcttcatcggccagcgcccccacgagcagctccccaccgccgactactgaaaacctcaatctcatctcgtccatccgtcatcgccatgagagattgaaatcgcttcaaaaattccagcaagtgtacgtcgtcgcgtcatcatcagtaagccagtttagtttcattgcgctatcttgtccttgagaaattcagggtaaacggtgtgtgcgagccagtcagtcaggaataatctcatccttctcttatcttggtgtatgtatgtacgtcctcaattatgaatgcttctattccatggtcaagattgatgtttgtacaaaaaaaaaaaaaaaaaa

SEQ ID NO: 23 - protein, 250 aa, Zea mays
MPINRIALGSHQEVYHPGALKAAFAEFISTLIFVFAGQGSGMAFSKLTGGGPTTPAGLIAAAVAHAFALFVAVSVGANISGGHVNPAVTFGAFVGGNITLFRGLLYWVAQLLGSTVACFLLRFSTGGQATGTFGLTGVSVWEALVLEIVMTFGLVYTVYATAVDPKKGSLGTIAPIAIGFIVGANILVGGAFDGASMNPAVSFGPALVSWEWGYQWVYWVGPLIGGGLAGVIYELLFISHTHEQLPSTDY